(19) United States
(12) Patent Application Publication
(10) Pub. No.: US 2025/0009712 A1
(43) Pub. Date: Jan. 9, 2025

(54) COMPOSITIONS AND METHODS OF TREATING ACNE AND PHOTOAGING

(71) Applicant: Curology, Inc., San Francisco, CA (US)

(72) Inventor: David Lortscher, San Francisco, CA (US)

(21) Appl. No.: 18/896,531

(22) Filed: Sep. 25, 2024

Related U.S. Application Data
(63) Continuation of application No. PCT/US2023/016212, filed on Mar. 24, 2023.
(60) Provisional application No. 63/269,931, filed on Mar. 25, 2022.

(51) Int. Cl. A61K 31/375; A61K 8/34; A61K 8/36; A61K 8/362; A61K 8/42; A61K 8/49; A61K 8/63; A61K 8/67; A61K 31/05; A61K 31/16; A61K 31/20; A61K 31/203; A61K 31/351; A61K 31/353; A61K 31/4164; A61K 31/455; A61K 31/555; A61K 31/573; A61K 31/58; A61K 31/7056; A61Q 19/08

(52) U.S. Cl. CPC A61K 31/375; A61K 8/347; A61K 8/361; A61K 8/362; A61K 8/42; A61K 8/4946; A61K 8/498; A61K 8/63; A61K 8/676; A61K 31/05; A61K 31/16; A61K 31/20; A61K 31/203; A61K 31/351; A61K 31/353; A61K 31/4164; A61K 31/455; A61K 31/555; A61K 31/573; A61K 31/58; A61K 31/7056; A61Q 19/08

(57) ABSTRACT

Pharmaceutical compositions for treating acne, photoaging, and uneven pigmentation comprising two or three distinct pharmaceutical ingredients are described. Methods for the treatment of acne, photoaging, and uneven pigmentation using the compositions are also described.

### RELATED APPLICATIONS

This application is a continuation of International Application No. PCT/US2023/016212, filed Mar. 24, 2023, which claims priority to U.S. Provisional Application No. 63/269,931, filed Mar. 25, 2022, the entire contents of which are incorporated herein by reference in their entireties.

### TECHNICAL FIELD

The present invention relates generally to the field of dermatology and more specifically to compositions and methods for the treatment of acne, uneven pigmentation, and photoaging.

### BACKGROUND

Acne occurs in greater than 90% of the population at some point in their lives. Although it is primarily considered a disorder of the teenage years, many people (and especially females) suffer from acne during adulthood. Acne (also known as acne vulgaris) is a long-term skin condition that is caused by: 1) plugging of hair follicles by abnormally keratinized cells; 2) microbial colonization of the follicle; 3) inflammation; and 4) increased oil production associated with circulating hormones.

Photoaging occurs naturally as our skin is exposed to the sun's ultraviolet rays, and the first signs of photoaging (including fine wrinkles and hyperpigmentation) typically appear between the ages of 20 and 35. While sun protection is key to minimizing photoaging, there are also various topical treatments which have proven to be efficacious for treating and preventing photoaging.

The desire to treat acne can coexist with the desire to treat and/or prevent photoaging. While the application of multiple dermatologic products is an option currently employed by many patients, the existence of a single, efficacious, stable composition would offer benefits in convenience and adherence.

However, there are numerous difficulties in formulating a single, efficacious, stable composition to treat or prevent acne and photoaging.

For example, the successful treatment of acne, alone, typically involves using two different agents with complementary mechanisms of action. The common categories are comedolytics (which help keratinization and thus prevent clogged pores) and antimicrobials (which generally target the acne-causing bacterium Propionibacterium acnes or P. acnes). Thus, the successful treatment of acne and photoaging together would typically require three or more active ingredients, which may require different vehicles, different frequencies of application, and different methods of application.

Another difficulty inherent in creating a combined formulation is that many anti-acne ingredients inactivate other anti-acne ingredients. For example, benzoyl peroxide inactivates tretinoin, erythromycin, and hydroquinone; tretinoin inactivates erythromycin; and benzoyl peroxide can lead to oxidation of zinc pyrithione. There are likely to be many more similar interactions that are not yet described in the dermatology literature.

When it is desired to use anti-photoaging ingredients or uneven pigmentation ingredients in addition to anti-acne ingredients, additional interactions can arise. When a patient is using benzoyl peroxide (for acne) they should avoid using it at the same time as hydroquinone (used for short-term treatment of photoaging), as the combination can lead to staining of the skin. As another example, niacinamide (a vitamin B3 derivative that can be used as an anti-acne ingredient and as an anti-photoaging ingredient) should not be used with ascorbic acid (the naturally occurring form of vitamin C), as the former can inactivate the latter ingredient. In addition, many photoaging or uneven pigmentation treatments cannot be used long-term because they contain steroids or a bleaching agent (hydroquinone) with potential undesirable side effects.

Thus, for patients receiving treatment for both acne and photoaging, their treatments typically are not included in the same formulation, and additionally, the patients are often instructed to use their individual formulations at different times of day, significantly decreasing the convenience of treatment.

An additional difficulty in formulating a once-daily composition for the treatment of acne and photoaging is that the majority of ingredients for each of these purposes are typically applied to the skin twice daily. These ingredients that are typically applied twice daily include clindamycin, azelaic acid, dapsone, adapalene, benzoyl peroxide, erythromycin, hydroquinone, niacinamide, ascorbic acid, magnesium ascorbyl phosphate, zinc pyrithione, and others. Even for treatment of acne alone, once-daily treatments are not yet the norm because of the potential inactivation of one anti-acne compound by another anti-acne compound and using two different agents with different mechanisms of action often requiring different formulations.

Also, a method to treat both acne and photoaging requires a collection of active ingredients that are stable and efficacious in the same vehicle. In formulating a vehicle of inactive ingredients to use along with active ingredient(s), one must account for texture, color, scent, method of application, pH, water solubility, alcohol solubility, stability of the active ingredients, and the presence or absence of interactions between the active ingredient(s) and the inactive ingredients. Thus, for both acne and photoaging to be treated with a single treatment is a significant advance over most current methodologies. A once-daily composition and method of treatment would be desirable because a once-daily composition increases patient adherence and lowers cost.

### SUMMARY

The disclosure provides pharmaceutical compositions, methods, and kits for the treatment of skin disorders, including acne and photoaging.

The disclosure provides a composition comprising: from 1% w/w to 10% w/w ascorbic acid and from 1% w/w to 15% w/w hydroquinone. The disclosure also provides a composition comprising: from 1% w/w to 10% w/w azelaic acid and from 0.1% w/w to 15% w/w hydroquinone. In some embodiments, these compositions further comprise one or more of: resveratrol, hydrocortisone, dexpanthenol, kojic acid, and epigallocatechin gallate. In some embodiments, the resveratrol is present in an amount from 0.1% w/w to 3% w/w. In some embodiments, the hydrocortisone is present in an amount from 0.5% w/w to 5% w/w. In some embodiments, the dexpanthenol is present in an amount from 0.1% w/w to 3% w/w. In some embodiments, the kojic acid is present in an amount from 2% w/w to 6% w/w. In some embodiments, the epigallocatechin gallate is present in an amount from 0.1% w/w to 6% w/w. In some embodiments, the epigallocatechin gallate is present in the form of green tea extract.

In some embodiments, the composition comprises about 4% w/w hydroquinone, about 3% w/w azelaic acid, and about 2.5% w/w hydrocortisone. In some embodiments, the composition comprises about 4% w/w hydroquinone, about 2.5% w/w hydrocortisone, and about 5% w/w ascorbic acid. In some embodiments, the composition further comprises about 4% w/w kojic acid.

In some embodiments, the composition comprises about 8% w/w hydroquinone, about 3% w/w azelaic acid, and about 2.5% w/w hydrocortisone. In some embodiments, the composition comprises about 8% w/w hydroquinone, about 2.5% w/w hydrocortisone, and about 5% w/w ascorbic acid. In some embodiments, the composition further comprises about 4% w/w kojic acid.

In some embodiments, the composition comprises about 12% w/w hydroquinone, about 3% w/w azelaic acid, and about 2.5% w/w hydrocortisone. In some embodiments, the composition comprises about 12% w/w hydroquinone, about 2.5% w/w hydrocortisone, and about 5% w/w ascorbic acid. In some embodiments, the composition further comprises about 4% w/w kojic acid.

In some embodiments, the composition comprises about 4% w/w hydroquinone, about 3% w/w azelaic acid, and about 1% w/w resveratrol. In some embodiments, the composition comprises about 4% w/w hydroquinone, about 1% w/w dexpanthenol, and about 5% w/w ascorbic acid. In some embodiments, the composition further comprises about 4% w/w kojic acid.

In some embodiments, the composition comprises about 8% w/w hydroquinone, about 3% w/w azelaic acid, and about 1% w/w resveratrol. In some embodiments, the composition comprises about 8% w/w hydroquinone, about 5% w/w ascorbic acid, and about 1% w/w dexpanthenol. In some embodiments, the composition further comprises about 4% w/w kojic acid.

In some embodiments, the composition comprises about 12% w/w hydroquinone, about 3% w/w azelaic acid, and about 1% w/w resveratrol. In some embodiments, the composition comprises about 12% w/w hydroquinone, about 5% w/w ascorbic acid, and about 1% w/w dexpanthenol. In some embodiments, the composition further comprises about 4% w/w kojic acid.

In some embodiments, the composition comprises about 4% w/w hydroquinone and about 5% w/w ascorbic acid. In some embodiments, the composition further comprises about 4% w/w kojic acid.

The disclosure also provides a composition comprising from 1% w/w to 10% w/w azelaic acid and from 0.1% w/w to 3% w/w resveratrol. In some embodiments, the composition further comprises one or more of: kojic acid, ascorbic acid, and epigallocatechin gallate. In some embodiments, the kojic acid is present in an amount from 2% w/w to 10% w/w. In some embodiments, the ascorbic acid is present in an amount from 1% w/w to 10% w/w. In some embodiments, the epigallocatechin gallate is present in the form of green tea extract.

In some embodiments, the composition comprises about 10% w/w azelaic acid and about 1% w/w resveratrol. In some embodiments, the composition comprises about 10% w/w azelaic acid, about 1% w/w resveratrol, and about 5% w/w ascorbic acid. In some embodiments, these compositions further comprise one or both of about 4% w/w kojic acid and about 2% w/w epigallocatechin gallate.

The disclosure also provides a composition comprising an anti-acne compound and a pharmaceutically acceptable vehicle, wherein the anti-acne compound consists of from 0.1% w/w to 5% w/w clindamycin and from 0.01% w/w to 1% w/w zinc pyrithione. In some embodiments, the anti-acne compound consists of about 1% w/w clindamycin and about 0.25% w/w zinc pyrithione.

The disclosure also provides a composition comprising an anti-acne compound and a pharmaceutically acceptable vehicle, wherein the anti-acne compound consists of from 1% w/w to 15% w/w azelaic acid and from 0.01% w/w to 1% w/w zinc pyrithione. In some embodiments, the anti-acne compound consists of about 3% w/w azelaic acid and about 0.25% w/w zinc pyrithione. In some embodiments, the anti-acne compound consists of about 6% w/w azelaic acid and about 0.25% w/w zinc pyrithione. In some embodiments, the anti-acne compound consists of about 10% w/w azelaic acid and about 0.25% w/w zinc pyrithione.

The disclosure also provides a composition comprising an anti-acne compound and a pharmaceutically acceptable vehicle, wherein the anti-acne compound consists of from 0.1% w/w to 5% w/w clindamycin and from 0.1% w/w to 15% w/w azelaic acid. In some embodiments, the anti-acne compound consists of about 1% w/w clindamycin and about 3% w/w azelaic acid. In some embodiments, the anti-acne compound consists of about 1% w/w clindamycin and about 6% w/w azelaic acid. In some embodiments, the anti-acne compound consists of about 1% w/w clindamycin and about 10% w/w azelaic acid.

The disclosure also provides a composition comprising an anti-acne compound and a pharmaceutically acceptable vehicle. In some embodiments, the anti-acne compound consists of from 0.1% w/w to 5% w/w clindamycin, from 0.1% w/w to 15% w/w azelaic acid, and from 0.01% w/w to 1% w/w zinc pyrithione. In some embodiments, the anti-acne compound consists of about 1% w/w clindamycin, about 2% w/w azelaic acid, and about 0.25% w/w zinc pyrithione.

The disclosure also provides a composition comprising an anti-acne compound and a pharmaceutically acceptable vehicle. In some embodiments, the anti-acne compound consists of from 0.1% w/w to 5% w/w clindamycin, from 0.1% w/w to 15% w/w azelaic acid, and from 1% w/w to 10% w/w niacinamide. In some embodiments, the anti-acne compound consists of about 1% w/w clindamycin, about 2% w/w azelaic acid, and about 4% w/w niacinamide.

The disclosure also provides a composition comprising: from 0.1% w/w to 15% w/w hydroquinone and from 0.005% w/w to 0.5% w/w desonide. In some embodiments, the composition comprises about 4% w/w hydroquinone and about 0.05% w/w desonide.

The disclosure also provides a composition comprising: from 0.1% w/w to 3% w/w metronidazole and from 1% w/w to 10% w/w niacinamide. In some embodiments, the composition further comprises azelaic acid. In some embodiments, the azelaic acid is present in an amount from 0.5% w/w to 20% w/w. In some embodiments, the composition comprises about 1% w/w metronidazole, about 4% w/w niacinamide, and about 2% w/w azelaic acid.

The disclosure also provides a composition comprising: from 0.5% w/w to 20% w/w azelaic acid and from 1% w/w to 10% w/w ascorbic acid. In some embodiments, the composition further comprises kojic acid. In some embodiments, the kojic acid is present in an amount of 2% to 6% w/w. In some embodiments, the composition comprises about 10% w/w azelaic acid and about 5% w/w ascorbic acid. In some embodiments, the composition further comprises about 4% w/w kojic acid.

The disclosure also provides a composition comprising: about 0.01% w/w tretinoin, about 1% w/w clindamycin, and about 2% w/w azelaic acid. The disclosure also provides a composition comprising: about 0.003% w/w tretinoin, about 1% w/w clindamycin, and about 2% w/w azelaic acid. The disclosure also provides a composition comprising: about 0.005% w/w tretinoin, about 1% w/w clindamycin, and about 2% w/w azelaic acid. The disclosure also provides a composition comprising: about 0.007% w/w tretinoin, about 1% w/w clindamycin, and about 2% w/w azelaic acid. The disclosure also provides a composition comprising: about 0.01% w/w tretinoin, about 1% w/w clindamycin, and about 4% w/w azelaic acid.

The disclosure also provides methods for treating or preventing acne in a subject in need thereof comprising administering to the skin of the subject any of the compositions described herein. The disclosure also provides methods for treating or preventing photoaging in a subject in need thereof comprising administering to the skin of the subject any of the compositions described herein. In some embodiments of these methods, the composition is administered once per day.

The disclosure also provides kits comprising any of the compositions described herein, a sealed container for housing the composition; and instructions for use.

### DETAILED DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

The disclosure provides pharmaceutical compositions, methods, and kits for the treatment of skin disorders.

### A. Terms, Definitions, and Abbreviations

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein and unless otherwise expressly noted or required by the context, all percentages refer to percentages by weight (wt-%) of the total composition (w/w).

As used herein in connection with a measured quantity, for example weight, “about” refers to that variation in the measured quantity as would be expected by one skilled in the art exercising a level of care commensurate with the objective of the measurement and the equipment used, and includes uncertainties that may be introduced by mathematical rounding errors. In certain embodiments, “about” means plus or minus 5% of the recited amount.

As used herein an “anti-acne” compound is a compound that treats acne, for example, reducing the amount of acne. Anti-acne compounds include, but are not limited to, comedolytics (which help keratinization and thus prevent clogged pores), antibiotics (which can target the acne-causing bacterium Propionibacterium acnes (P. acnes) or the acne-causing mite Demodex folliculorum), phenols (e.g., epigallocatechin gallate, and resveratrol), chelating agents (e.g., kojic acid), and anti-inflammatory compounds (which have a direct effect on inflammation independent of any comedolytic or antibiotic effects). Non-limiting examples of comedolytics include alpha hydroxy acids (e.g., glycolic acid, lactic acid, and salicylic acid), retinoids (e.g., tretinoin and isotretinoin), and saturated dicarboxylic acids (e.g., suberic acid, azelaic acid, and sebacic acid). Non-limiting examples of antibiotics include cephalosporins (e.g., cefoxitin, ceftazidime, and cefepime), nitroimidazole (e.g., metronidazole), lincosamides (e.g., clindamycin and lincomycin), macrolides (e.g., erythromycin and azithromycin), pleuromutilins (e.g., retapamulin), metal complexes (e.g., zinc pyrithione, zinc methoxazole, and zinc sulfathiazole), penicillins (e.g., amoxicillin, ampicillin, and carbenicillin), fluoroquinolones (e.g., ciprofloxacin, clinafloxacin, ofloxacin, and trovafloxacin), retinoids (e.g., tretinoin), saturated dicarboxylic acids (e.g., suberic acid, azelaic acid, and sebacic acid), sulfonamides (e.g., sulfamethizole, sulfamethoxazole, sulfisoxazole), sulfones (e.g., dapsone or diaminodiphenyl sulfone), and tetracyclines (e.g., doxycycline and minocycline). Non-limiting examples of an anti-inflammatory compound include lincosamides (e.g., clindamycin and lincomycin), niacinamide (also known as nicotinamide and pyridine-3-corboxamide), retinoids (e.g., tretinoin), saturated dicarboxylic acids (e.g., suberic acid, azelaic acid, and sebacic acid), corticosteroids (e.g., desonide), and hydrocortisone.

Saturated dicarboxylic acids can act as comedolytics and as antibiotics. Although azelaic acid is a useful anti-acne compound for use in those embodiments of the present disclosure in which an anti-acne compound is included, other saturated dicarboxylic acids may also be used, including suberic acid and sebacic acid. Azelaic acid (also known as nonanedioic acid) is an external treatment for, for example, acne, rosacea, melasma, and postinflammatory hyperpigmentation. Azelaic acid is also used as an antifungal.

Nitroimidazoles can act as antibiotics and anti-inflammatory agents. For example, metronidazole exhibits both antibiotic and anti-inflammatory activity. Metronidazole has antibiotic activity against Demodex folliculorum, a mite that lives on the skin that may play a role in both acne and rosacea.

Retinoids are well known to those skilled in the art of formulating topical dermatological compositions. Retinoids exhibit the pharmacological activity of all trans retinol and share, as a common structural feature, a β-ionone-type ring (2,6,6-trimethylcyclohen-1-ene) having a multiply unsaturated alkyl side chain at the 1 position of the ring. Tretinoin (also known all-trans retinoic acid) is the carboxylic acid form of vitamin A. Tretinoin, as well as other retinoid derivatives, such as but not limited to, retinol, adapalene, isotretinoin, alitretinoin, etretinate, acitretin, bexarotene, or tazarotene can also be used as anti-acne or anti-aging compounds.

Some metal complexes, including zinc pyrithione, have antibiotic effects. Although zinc pyrithione (also known as bis(2-pyridylthio) zinc 1,1′-dioxide) is a useful anti-acne compound for use in those embodiments of the present disclosure in which an anti-acne compound is included, other metal complexes can also be used, including zinc methoxazole and zinc sulfathiazole. Zinc pyrithione is also used as an antifungal. Zinc pyrithione is used to treat and prevent UV-induced skin damage and may also treat hyperpigmentation such as melasma.

The term “anti-inflammatory” compound for the purposes of the present disclosure refers to a compound that reduces certain signs of inflammation and may treat inflammatory acne (e.g., papules, pustules, nodules, and cysts) independent of any comedolytic or antimicrobial effects. For example, anti-inflammatory compounds include azelaic acid, niacinamide, tretinoin, desonide, and hydrocortisone.

As used herein an “anti-photoaging” compound is a compound that treats photoaging, for example, by reducing the amount of fine wrinkles or of hyperpigmentation. Anti-photoaging compounds include, but are not limited to, antioxidants (e.g., vitamins or vitamin derivatives including, but not limited to, niacinamide and ascorbyl phosphate, ascorbyl 6 palmitate, isostearyl 2-O L-ascorbyl phosphate, ascorbic acid sulfate, and dexpanthenol), tyrosinase inhibitors (e.g., 4-n-butylresorcinol, azelaic acid, and kojic acid), and skin lightening agents (e.g., tranexamic acid and hydroquinone). Tranexamic acid has skin lightening effects and can be used to treat hyperpigmentation, including hyperpigmentation that results from exposure to ultraviolet light and melasma.

The term “antioxidant” for the purposes of the present disclosure refers to a chemical substance that is added to a pharmaceutical composition to treat or to prevent photoaging, for example, by inhibiting the oxidation of molecules that are present in skin or dermis of a subject. Vitamins and vitamin derivatives are well known to those skilled in the art of formulating topical dermatological compositions. Certain vitamin derivatives have increased stability over the naturally occurring form of the vitamin. For example, some vitamin E derivatives, including tocopheryl acetate, are more stable than the naturally occurring tocopherol (vitamin E), and some vitamin C derivatives, including ascorbyl phosphate, ascorbyl 6 palmitate, isostearyl 2-O L-ascorbyl phosphate, and ascorbic acid sulfate, or pharmaceutically acceptable salts thereof, are more stable than the naturally occurring L-ascorbic acid or ascorbate (vitamin C). Vitamin C is the most abundant antioxidant in the skin and is a cofactor in collagen production. Niacinamide is a form of vitamin B3 that fights acne via anti-inflammatory properties and has anti-aging effects.

The term “tyrosinase inhibitor” for the purposes of the present disclosure refers to a chemical compound that is added to a pharmaceutical composition to treat or to prevent photoaging, for example, by reducing the production of melanin by binding to tyrosinase present in skin or dermis of a subject. Tyrosinase is a copper-containing oxidase that catalyzes the first two steps in the production of melanin. Overproduction of melanin can lead to hyperpigmentation. Although azelaic acid is a useful anti-photoaging compound for use in those embodiments of the present disclosure in which an anti-photoaging compound is included, other tyrosinase inhibitors can also be used, including 4-n-butylresorcinol and kojic acid.

An “inactive ingredient” is compatible with the other ingredients of the formulation and not injurious to the patient or to the subject. Non-limiting examples of inactive ingredients include a preservative, a thickening agent, a vehicle, and a vitamin derivative.

The term “preservative” for the purposes of the present invention refers to a chemical substance that is added to a pharmaceutical composition to prevent the pharmaceutical composition from deterioration, decomposition, or degradation or to substantially reduce or decelerate the degree and/or the speed of such deterioration, decomposition, or degradation. Non-limiting examples of preservatives include benzoate, ethylhexylglycerin, methyl benzoate, methyl paraben, phenoxyethanol, propionic acid, propyl paraben, and pharmaceutically acceptable salts thereof.

An antioxidant can also be a preservative that contributes to the long-term storage and stability of the composition because of its function as a free radical scavenger. Non-limiting examples of antioxidant preservatives include butylated hydroxytoluene (BHT), butylated hydroxyanisole (BHA), tocopheryl acetate, ascorbic acid, ascorbyl palmitate, lecithin, nordihydroguaiaretic acid, propyl gallate, α-tocopherol, sodium bisulfite, cysteine, sodium metabisulfite, thioglycerol, thioglycolic acid, thiomersal, and pharmaceutically acceptable salts thereof.

The term “vehicle” refers to a substance that serves as a carrier, whether diluent or excipient, for improving the efficiency of delivery and the effectiveness of a pharmaceutical composition. The phrase “pharmaceutically acceptable vehicle” is art recognized and includes a pharmaceutically acceptable material, composition, or vehicle suitable for administering compounds of the present invention to mammals. The vehicles include liquid or solid filler, diluent, excipient, solvent, or encapsulating material, involved in carrying or transporting the subject agent from one organ, or portion of the body, to another organ, or portion of the body. Each vehicle must be “acceptable” in the sense of being compatible with the other ingredients of the formulation and not injurious to the patient or to the subject. Some examples of materials which can serve as pharmaceutically acceptable vehicles include: water; aloe vera leaf juice; aloe barbadensis leaf juice; emulsifiers or thickening agents, such as carbomer, cetearyl alcohol, cetyl alcohol, glyceryl stearate, stearic acid, xanthan gum, C13-14 isoparaffin, caprylic/capric triglyceride, polyacrylamide, and viscous liquids; sugars, such as lactose, glucose and sucrose; starches, such as corn starch and potato starch; maltodextrin; cellulose, and its derivatives, such as sodium carboxymethyl cellulose, ethyl cellulose and cellulose acetate; powdered tragacanth; malt; gelatin; talc; excipients, such as cocoa butter, myristyl myristate, Shea butter, and suppository waxes; oils, such as acai palm fruit oil, calendula flower oil, corn oil, cottonseed oil, jojoba seed oil, olive oil, passion fruit seed oil, peanut oil, rice bran oil, safflower oil, sesame oil, soybean oil, and sweet almond seed oil; glycols, such as propylene glycol and tricthylene glycol; polyols, such as glycerin, vegetable glycerin, sorbitol, mannitol and polyethylene glycol (e.g., ceteareth-20, laureth-7, and PEG-100 myristate); esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As used herein, the term “pharmaceutically acceptable salts” refers to derivatives of the disclosed compounds, wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are useful. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### B. Compositions

The disclosure provides pharmaceutical compositions for the treatment of skin disorders, such as a topically administered compositions. In some aspects, the pharmaceutical compositions comprise two distinct pharmaceutical ingredients, which may be supplied in a single topical pharmaceutical composition. In some aspects, the pharmaceutical compositions comprise two or three distinct pharmaceutical ingredients, which may be supplied in a single topical pharmaceutical composition. The two or three ingredients are selected from the following:

In some embodiments, the composition comprises niacinamide, or a pharmaceutically acceptable salt thereof, and can range, e.g., from about 0.1% w/w to about 5.0% w/w of the composition. In some embodiments, niacinamide is about 0.1%, about 0.5%, about 1.0%, about 1.5%, about 2.0% w/w, about 2.0%, about 2.5%, about 3.0%, about 3.5%, about 4.0%, about 4.5%, or about 5.0% of the composition. In some embodiments, the niacinamide, or a pharmaceutically acceptable salt thereof, is about 4% w/w of the composition.

In some embodiments, the composition comprises niacinamide, or a pharmaceutically acceptable salt thereof, and can range, e.g., from 0.1% w/w to 5.0% w/w of the composition. In some embodiments, niacinamide is 0.1%, 0.5%, 1.0%, 1.5%, 2.0% w/w, 2.0%, 2.5%, 3.0%, 3.5%, 4.0%, 4.5%, or 5.0% of the composition. In some embodiments, the niacinamide, or a pharmaceutically acceptable salt thereof, is 4% w/w of the composition.

In some embodiments, the composition comprises tretinoin, or a pharmaceutically acceptable salt thereof, and can range, e.g., from about 0.001% to about 1% w/w of the composition, from about 0.001% to about 0.2% w/w of the composition, or is about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.12%, about 0.13%, about 0.14%, or about 0.15% w/w of the composition. In other embodiments, the tretinoin, or a pharmaceutically acceptable salt thereof, is about 0.003%, about 0.005%, about 0.007%, or about 0.010% w/w of the composition. In some embodiments comprising tretinoin, the composition further comprises the antioxidant butylated hydroxytoluene (BHT).

In some embodiments, the composition comprises tretinoin, or a pharmaceutically acceptable salt thereof, and can range, e.g., from 0.001% to 1% w/w of the composition, from 0.001% to 0.2% w/w of the composition, or is 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.12%, 0.13%, 0.14%, or 0.15% w/w of the composition. In other embodiments, the tretinoin, or a pharmaceutically acceptable salt thereof, is 0.003%, 0.005%, 0.007%, or 0.010% w/w of the composition. In some embodiments comprising tretinoin, the composition further comprises the antioxidant butylated hydroxytoluene (BHT).

In some embodiments, the composition comprises zinc pyrithione, or a pharmaceutically acceptable salt thereof, and can range, e.g., from about 0.01% to about 1% w/w of the composition or is about 0.1%, about 0.15%, about 0.2%, about 0.25%, about 0.3%, about 0.35%, about 0.4%, about 0.45%, about 0.5%, about 0.55%, about 0.60%, about 0.65%, about 0.70%, about 0.75%, about 0.80%, about 0.85%, about 0.90%, about 0.95%, or about 1.0% w/w of the composition. In some embodiments the zinc pyrithione, or a pharmaceutically acceptable salt thereof, is about 0.25% w/w of the composition.

In some embodiments, the composition comprises zinc pyrithione, or a pharmaceutically acceptable salt thereof, and can range, e.g., from 0.01% to 1% w/w of the composition or is 0.1%, 0.15%, 0.2%, 0.25%, 0.3%, 0.35%, 0.4%, 0.45%, 0.5%, 0.55%, 0.60%, 0.65%, 0.70%, 0.75%, 0.80%, 0.85%, 0.90%, 0.95%, or 1.0% w/w of the composition. In some embodiments the zinc pyrithione, or a pharmaceutically acceptable salt thereof, is 0.25% w/w of the composition.

In some embodiments, the composition comprises azelaic acid, or a pharmaceutically acceptable salt thereof, and can range, e.g., from about 0.1% w/w to about 15% w/w of the composition, from about 0.5% w/w to about 20% w/w of the composition, from about 1% w/w to about 10% w/w of the composition, from about 1% w/w to about 15% w/w of the composition, or is about 2%, about 3%, about 4%, about 5%, about 6%, about 7%, about 8%, about 9%, or about 10% w/w of the composition. In some embodiments, the azelaic acid, or a pharmaceutically acceptable salt thereof, is about 2% w/w of the composition. In some embodiments, the azelaic acid, or a pharmaceutically acceptable salt thereof, is about 3% w/w of the composition. In some embodiments, the azelaic acid, or a pharmaceutically acceptable salt thereof, is about 6% w/w of the composition. In some embodiments, the azelaic acid, or a pharmaceutically acceptable salt thereof, is about 10% w/w of the composition.

In some embodiments, the composition comprises azelaic acid, or a pharmaceutically acceptable salt thereof, and can range, e.g., from 0.1% w/w to 15% w/w of the composition, from 0.5% w/w to 20% w/w of the composition, from 1% w/w to 10% w/w of the composition, from 1% w/w to 15% w/w of the composition, or is 2%, 3%, 4%, 5%, 6%, 7%, 8%, 9%, or 10% w/w of the composition. In some embodiments, the azelaic acid, or a pharmaceutically acceptable salt thereof, is 2% w/w of the composition. In some embodiments, the azelaic acid, or a pharmaceutically acceptable salt thereof, is 3% w/w of the composition. In some embodiments, the azelaic acid, or a pharmaceutically acceptable salt thereof, is 6% w/w of the composition. In some embodiments, the azelaic acid, or a pharmaceutically acceptable salt thereof, is 10% w/w of the composition.

In some embodiments, the composition comprises ascorbic acid, or a pharmaceutically acceptable salt thereof, and can range, e.g., from about 1% w/w to about 10% w/w of the composition, from about 2% w/w to about 7% w/w of the composition, or is about 2%, about 3%, about 4%, about 5%, about 6%, about 7%, or about 8% w/w of the composition. In some embodiments, the ascorbic acid, or a pharmaceutically acceptable salt thereof, is about 5% w/w of the composition.

In some embodiments, the composition comprises ascorbic acid, or a pharmaceutically acceptable salt thereof, and can range, e.g., from 1% w/w to 10% w/w of the composition, from 2% w/w to 7% w/w of the composition, or is 2%, 3%, 4%, 5%, 6%, 7%, or 8% w/w of the composition. In some embodiments, the ascorbic acid, or a pharmaceutically acceptable salt thereof, is 5% w/w of the composition.

In some embodiments, the composition comprises hydroquinone, or a pharmaceutically acceptable salt thereof, and can range, e.g., from about 0.1% w/w to about 15% w/w of the composition, from about 1% w/w to about 15% w/w of the composition, or is about 2%, about 3%, about 4%, about 5%, about 6%, about 7%, about 8%, about 9%, about 10%, about 11%, or about 12% w/w of the composition. In some embodiments, the hydroquinone, or a pharmaceutically acceptable salt thereof, is about 4% w/w of the composition. In some embodiments, the hydroquinone, or a pharmaceutically acceptable salt thereof, is about 8% w/w of the composition. In some embodiments, the hydroquinone, or a pharmaceutically acceptable salt thereof, is about 12% w/w of the composition.

In some embodiments, the composition comprises hydroquinone, or a pharmaceutically acceptable salt thereof, and can range, e.g., from 0.1% w/w to 15% w/w of the composition, from 1% w/w to 15% w/w of the composition, or is 2%, 3%, 4%, 5%, 6%, 7%, 8%, 9%, 10%, 11%, or 12% w/w of the composition. In some embodiments, the hydroquinone, or a pharmaceutically acceptable salt thereof, is 4% w/w of the composition. In some embodiments, the hydroquinone, or a pharmaceutically acceptable salt thereof, is 8% w/w of the composition. In some embodiments, the hydroquinone, or a pharmaceutically acceptable salt thereof, is 12% w/w of the composition.

In some embodiments, the composition comprises resveratrol, or a pharmaceutically acceptable salt thereof, and can range, e.g., from about 0.1% w/w to about 3.0% w/w of the composition, or is about 0.1%, about 0.5%, about 1.0%, about 1.5%, about 2.0%, about 2.5%, or about 3.0% w/w of the composition. In some embodiments, the resveratrol, or a pharmaceutically acceptable salt thereof, is about 1% w/w of the composition.

In some embodiments, the composition comprises resveratrol, or a pharmaceutically acceptable salt thereof, and can range, e.g., from 0.1% w/w to 3.0% w/w of the composition, or is 0.1%, 0.5%, 1.0%, 1.5%, 2.0%, 2.5%, or 3.0% w/w of the composition. In some embodiments, the resveratrol, or a pharmaceutically acceptable salt thereof, is 1% w/w of the composition.

In some embodiments, the composition comprises hydrocortisone, or a pharmaceutically acceptable salt thereof, and can range, e.g., from about 0.5% w/w to about 5.0% w/w of the composition, or is about 0.5%, about 1.0%, about 1.5%, about 2.0%, about 2.5%, about 3.0% w/w, about 3.5% w/w, about 4.5% w/w, about 5.0% w/w of the composition. In some embodiments, the hydrocortisone, or a pharmaceutically acceptable salt thereof, is about 2.5% w/w of the composition.

In some embodiments, the composition comprises hydrocortisone, or a pharmaceutically acceptable salt thereof, and can range, e.g., from 0.5% w/w to 5.0% w/w of the composition, or is 0.5%, 1.0%, 1.5%, 2.0%, 2.5%, 3.0% w/w, 3.5% w/w, 4.5% w/w, 5.0% w/w of the composition. In some embodiments, the hydrocortisone, or a pharmaceutically acceptable salt thereof, is 2.5% w/w of the composition.

In some embodiments, the composition comprises dexpanthenol, or a pharmaceutically acceptable salt thereof, and can range, e.g., from about 0.1% w/w to about 3.0% w/w of the composition, or is about 0.1%, about 0.5%, about 1.0%, about 1.5%, about 2.0%, about 2.5%, or about 3.0% w/w of the composition. In some embodiments, the dexpanthenol, or a pharmaceutically acceptable salt thereof, is about 1.0% w/w of the composition.

In some embodiments, the composition comprises dexpanthenol, or a pharmaceutically acceptable salt thereof, and can range, e.g., from 0.1% w/w to 3.0% w/w of the composition, or is 0.1%, 0.5%, 1.0%, 1.5%, 2.0%, 2.5%, or 3.0% w/w of the composition. In some embodiments, the dexpanthenol, or a pharmaceutically acceptable salt thereof, is 1.0% w/w of the composition.

In some embodiments, the composition comprises kojic acid, or a pharmaceutically acceptable salt thereof, and can range, e.g., from about 2% w/w to about 10% w/w, from about 2% w/w to about 6% w/w of the composition, or is about 2%, about 3%, about 4%, about 5%, about 6%, about 7%, about 8%, about 9% w/w, or about 10% w/w of the composition. In some embodiments, the kojic acid, or a pharmaceutically acceptable salt thereof, is about 4% w/w of the composition.

In some embodiments, the composition comprises kojic acid, or a pharmaceutically acceptable salt thereof, and can range, e.g., from 2% w/w to 10% w/w, from 2% w/w to 6% w/w of the composition, or is 2%, 3%, 4%, 5%, 6%, 7%, 8%, 9% w/w, or 10% w/w of the composition. In some embodiments, the kojic acid, or a pharmaceutically acceptable salt thereof, is 4% w/w of the composition.

In some embodiments, the composition comprises epigallocatechin gallate, or a pharmaceutically acceptable salt thereof, and can range, e.g., from about 0.1% w/w to about 6.0% w/w of the composition, or is about 0.1%, about 0.5%, about 1.0%, about 1.5%, about 2.0%, about 2.5%, about 3.0%, about 3.5% w/w, about 4.0% w/w, about 4.5% w/w, about 5.0% w/w, about 5.5% w/w, or about 6.0% w/w of the composition. In some embodiments, the epigallocatechin gallate, or a pharmaceutically acceptable salt thereof, is about 2.0% w/w of the composition.

In some embodiments, the composition comprises epigallocatechin gallate, or a pharmaceutically acceptable salt thereof, and can range, e.g., from 0.1% w/w to 6.0% w/w of the composition, or is 0.1%, 0.5%, 1.0%, 1.5%, 2.0%, 2.5%, 3.0%, 3.5% w/w, 4.0% w/w, 4.5% w/w, 5.0% w/w, 5.5% w/w, or 6.0% w/w of the composition. In some embodiments, the epigallocatechin gallate, or a pharmaceutically acceptable salt thereof, is 2.0% w/w of the composition.

In some embodiments, the composition comprises clindamycin, or a pharmaceutically acceptable salt thereof, and can range, e.g., from about 0.1% w/w to about 5.0% w/w of the composition, or is about 0.1%, about 0.5%, about 1.0%, about 1.5%, about 2.0%, about 2.5%, about 3.0%, about 3.5% w/w, about 4.0% w/w, about 4.5%, or about 5.0% w/w of the composition. In some embodiments, the clindamycin, or a pharmaceutically acceptable salt thereof, is about 1.0% w/w of the composition.

In some embodiments, the composition comprises clindamycin, or a pharmaceutically acceptable salt thereof, and can range, e.g., from 0.1% w/w to 5.0% w/w of the composition, or is 0.1%, 0.5%, 1.0%, 1.5%, 2.0%, 2.5%, 3.0%, 3.5% w/w, 4.0% w/w, 4.5%, or 5.0% w/w of the composition. In some embodiments, the clindamycin, or a pharmaceutically acceptable salt thereof, is 1.0% w/w of the composition.

In some embodiments, the composition comprises desonide, or a pharmaceutically acceptable salt thereof, and can range, e.g., from about 0.005% w/w to about 0.5% w/w of the composition, or is about 0.005%, about 0.01%, about 0.05%, about 0.1%, about 0.2%, about 0.3%, about 0.4%, or about 0.5% w/w of the composition. In some embodiments, the desonide, or a pharmaceutically acceptable salt thereof, is about 0.05% w/w of the composition.

In some embodiments, the composition comprises desonide, or a pharmaceutically acceptable salt thereof, and can range, e.g., from 0.005% w/w to 0.5% w/w of the composition, or is 0.005%, 0.01%, 0.05%, 0.1%, 0.2%, 0.3%, 0.4%, or 0.5% w/w of the composition. In some embodiments, the desonide, or a pharmaceutically acceptable salt thereof, is 0.05% w/w of the composition.

In some embodiments, the composition comprises metronidazole, or a pharmaceutically acceptable salt thereof, and can range, e.g., from about 0.1% w/w to about 3.0% w/w of the composition, or is about 0.1%, about 0.5%, about 1.0%, about 2.0%, or about 3.0% w/w of the composition. In some embodiments, the metronidazole, or a pharmaceutically acceptable salt thereof, is about 1.0% w/w of the composition.

In some embodiments, the composition comprises metronidazole, or a pharmaceutically acceptable salt thereof, and can range, e.g., from 0.1% w/w to 3.0% w/w of the composition, or is 0.1%, 0.5%, 1.0%, 2.0%, or 3.0% w/w of the composition. In some embodiments, the metronidazole, or a pharmaceutically acceptable salt thereof, is 1.0% w/w of the composition.

In some embodiments, the composition comprises ascorbic acid and hydroquinone, or pharmaceutically acceptable salts thereof. In some embodiments, the composition consists of ascorbic acid and hydroquinone, or pharmaceutically acceptable salts thereof. In some embodiments, the composition comprises two compounds comprising from 1% w/w to 10% w/w ascorbic acid and from 1% w/w to 15% w/w hydroquinone. In some embodiments, the composition further comprises one or more of: resveratrol, hydrocortisone, dexpanthenol, kojic acid, and epigallocatechin gallate. In some embodiments, the composition comprises from 0.1% w/w to 3% w/w resveratrol. In some embodiments, the composition comprises from 0.5% w/w to 5% w/w hydrocortisone. In some embodiments, the composition comprises from 0.1% w/w to 3% w/w dexpanthenol. In some embodiments, the composition comprises from 2% w/w to 6% w/w kojic acid. In some embodiments, the composition comprises from 0.1% w/w to 6% w/w epigallocatechin gallate. In some embodiments, the composition comprises epigallocatechin gallate in the form of green tea extract.

In some embodiments, the composition comprises about 4% w/w hydroquinone, about 2.5% w/w hydrocortisone, and about 5% w/w ascorbic acid. In some embodiments, the composition comprises about 4% w/w hydroquinone, about 2.5% w/w hydrocortisone, about 5% w/w ascorbic acid, and about 4% w/w kojic acid.

In some embodiments, the composition comprises 4% w/w hydroquinone, 2.5% w/w hydrocortisone, and 5% w/w ascorbic acid. In some embodiments, the composition comprises 4% w/w hydroquinone, 2.5% w/w hydrocortisone, 5% w/w ascorbic acid, and 4% w/w kojic acid.

In some embodiments, the composition comprises an anti-acne compound and a pharmaceutically acceptable vehicle, wherein the anti-acne compound consists of about 4% w/w hydroquinone, about 2.5% w/w hydrocortisone, and about 5% w/w ascorbic acid. In some embodiments, the anti-acne compound consists of about 4% w/w hydroquinone, about 2.5% w/w hydrocortisone, about 5% w/w ascorbic acid, and about 4% w/w kojic acid.

In some embodiments, the composition comprises an anti-acne compound and a pharmaceutically acceptable vehicle, wherein the anti-acne compound consists of 4% w/w hydroquinone, 2.5% w/w hydrocortisone, and 5% w/w ascorbic acid. In some embodiments, the anti-acne compound consists of 4% w/w hydroquinone, 2.5% w/w hydrocortisone, 5% w/w ascorbic acid, and 4% w/w kojic acid.

In some embodiments, the composition comprises about 8% w/w hydroquinone, about 2.5% w/w hydrocortisone, and about 5% w/w ascorbic acid. In some embodiments, the composition comprises about 8% w/w hydroquinone, about 2.5% w/w hydrocortisone, about 5% w/w ascorbic acid, and about 4% w/w kojic acid.

In some embodiments, the composition comprises 8% w/w hydroquinone, 2.5% w/w hydrocortisone, and 5% w/w ascorbic acid. In some embodiments, the composition comprises 8% w/w hydroquinone, 2.5% w/w hydrocortisone, 5% w/w ascorbic acid, and 4% w/w kojic acid.

In some embodiments, the composition comprises an anti-acne compound and a pharmaceutically acceptable vehicle, wherein the anti-acne compound consists of about 8% w/w hydroquinone, about 2.5% w/w hydrocortisone, and about 5% w/w ascorbic acid. In some embodiments, the anti-acne compound consists of about 8% w/w hydroquinone, about 2.5% w/w hydrocortisone, about 5% w/w ascorbic acid, and about 4% w/w kojic acid.

In some embodiments, the composition comprises an anti-acne compound and a pharmaceutically acceptable vehicle, wherein the anti-acne compound consists of 8% w/w hydroquinone, 2.5% w/w hydrocortisone, and 5% w/w ascorbic acid. In some embodiments, the anti-acne compound consists of 8% w/w hydroquinone, 2.5% w/w hydrocortisone, 5% w/w ascorbic acid, and 4% w/w kojic acid.

In some embodiments, the composition comprises about 12% w/w hydroquinone, about 2.5% w/w hydrocortisone, and about 5% w/w ascorbic acid. In some embodiments, the composition comprises about 12% w/w hydroquinone, about 2.5% w/w hydrocortisone, about 5% w/w ascorbic acid, and about 4% w/w kojic acid.

In some embodiments, the composition comprises 12% w/w hydroquinone, 2.5% w/w hydrocortisone, and 5% w/w ascorbic acid. In some embodiments, the composition comprises 12% w/w hydroquinone, 2.5% w/w hydrocortisone, 5% w/w ascorbic acid, and 4% w/w kojic acid.

In some embodiments, the composition comprises an anti-acne compound and a pharmaceutically acceptable vehicle, wherein the anti-acne compound consists of about 12% w/w hydroquinone, about 2.5% w/w hydrocortisone, and about 5% w/w ascorbic acid. In some embodiments, the anti-acne compound consists of about 12% w/w hydroquinone, about 2.5% w/w hydrocortisone, about 5% w/w ascorbic acid, and about 4% w/w kojic acid.

In some embodiments, the composition comprises an anti-acne compound and a pharmaceutically acceptable vehicle, wherein the anti-acne compound consists of 12% w/w hydroquinone, 2.5% w/w hydrocortisone, and 5% w/w ascorbic acid. In some embodiments, the anti-acne compound consists of 12% w/w hydroquinone, 2.5% w/w hydrocortisone, 5% w/w ascorbic acid, and 4% w/w kojic acid.

In some embodiments, the composition comprises about 4% w/w hydroquinone, about 1% w/w dexpanthenol, and about 5% w/w ascorbic acid. In some embodiments, the composition comprises about 4% w/w hydroquinone, about 1% w/w dexpanthenol, about 5% w/w ascorbic acid, and about 4% w/w kojic acid.

In some embodiments, the composition comprises 4% w/w hydroquinone, 1% w/w dexpanthenol, and 5% w/w ascorbic acid. In some embodiments, the composition comprises 4% w/w hydroquinone, 1% w/w dexpanthenol, 5% w/w ascorbic acid, and 4% w/w kojic acid.

In some embodiments, the composition comprises an anti-acne compound and a pharmaceutically acceptable vehicle, wherein the anti-acne compound consists of about 4% w/w hydroquinone, about 1% w/w dexpanthenol, and about 5% w/w ascorbic acid. In some embodiments, the anti-acne compound consists of about 4% w/w hydroquinone, about 1% w/w dexpanthenol, about 5% w/w ascorbic acid, and about 4% w/w kojic acid.

In some embodiments, the composition comprises an anti-acne compound and a pharmaceutically acceptable vehicle, wherein the anti-acne compound consists of 4% w/w hydroquinone, 1% w/w dexpanthenol, and 5% w/w ascorbic acid. In some embodiments, the anti-acne compound consists of 4% w/w hydroquinone, 1% w/w dexpanthenol, 5% w/w ascorbic acid, and 4% w/w kojic acid.

In some embodiments, the composition comprises about 8% w/w hydroquinone, about 1% w/w dexpanthenol, and about 5% w/w ascorbic acid. In some embodiments, the composition comprises about 8% w/w hydroquinone, about 1% w/w dexpanthenol, about 5% w/w ascorbic acid, and about 4% w/w kojic acid.

In some embodiments, the composition comprises 8% w/w hydroquinone, 1% w/w dexpanthenol, and 5% w/w ascorbic acid. In some embodiments, the composition comprises 8% w/w hydroquinone, 1% w/w dexpanthenol, 5% w/w ascorbic acid, and 4% w/w kojic acid.

In some embodiments, the composition comprises an anti-acne compound and a pharmaceutically acceptable vehicle, wherein the anti-acne compound consists of about 8% w/w hydroquinone, about 1% w/w dexpanthenol, and about 5% w/w ascorbic acid. In some embodiments, the anti-acne compound consists of about 8% w/w hydroquinone, about 1% w/w dexpanthenol, about 5% w/w ascorbic acid, and about 4% w/w kojic acid.

In some embodiments, the composition comprises an anti-acne compound and a pharmaceutically acceptable vehicle, wherein the anti-acne compound consists of 8% w/w hydroquinone, 1% w/w dexpanthenol, and 5% w/w ascorbic acid. In some embodiments, the anti-acne compound consists of 8% w/w hydroquinone, 1% w/w dexpanthenol, 5% w/w ascorbic acid, and 4% w/w kojic acid.

In some embodiments, the composition comprises about 12% w/w hydroquinone, about 1% w/w dexpanthenol, and about 5% w/w ascorbic acid. In some embodiments, the composition comprises about 12% w/w hydroquinone, about 1% w/w dexpanthenol, about 5% w/w ascorbic acid, and about 4% w/w kojic acid.

In some embodiments, the composition comprises 12% w/w hydroquinone, 1% w/w dexpanthenol, and 5% w/w ascorbic acid. In some embodiments, the composition comprises 12% w/w hydroquinone, 1% w/w dexpanthenol, 5% w/w ascorbic acid, and 4% w/w kojic acid.

In some embodiments, the composition comprises an anti-acne compound and a pharmaceutically acceptable vehicle, wherein the anti-acne compound consists of about 12% w/w hydroquinone, about 1% w/w dexpanthenol, and about 5% w/w ascorbic acid. In some embodiments, the anti-acne compound consists of 12% w/w hydroquinone, about 1% w/w dexpanthenol, about 5% w/w ascorbic acid, and about 4% w/w kojic acid.

In some embodiments, the composition comprises an anti-acne compound and a pharmaceutically acceptable vehicle, wherein the anti-acne compound consists of 12% w/w hydroquinone, 1% w/w dexpanthenol, and 5% w/w ascorbic acid. In some embodiments, the anti-acne compound consists of 12% w/w hydroquinone, 1% w/w dexpanthenol, 5% w/w ascorbic acid, and 4% w/w kojic acid.

In some embodiments, the composition comprises about 4% w/w hydroquinone, and about 5% w/w ascorbic acid. In some embodiments, the composition comprises about 4% w/w hydroquinone, about 5% w/w ascorbic acid, and about 4% w/w kojic acid.

In some embodiments, the composition comprises 4% w/w hydroquinone, and 5% w/w ascorbic acid. In some embodiments, the composition comprises 4% w/w hydroquinone, 5% w/w ascorbic acid, and 4% w/w kojic acid.

In some embodiments, the composition comprises an anti-acne compound and a pharmaceutically acceptable vehicle, wherein the anti-acne compound consists of about 4% w/w hydroquinone, and about 5% w/w ascorbic acid. In some embodiments, the anti-acne compound consists of about 4% w/w hydroquinone, about 5% w/w ascorbic acid, and about 4% w/w kojic acid.

In some embodiments, the composition comprises an anti-acne compound and a pharmaceutically acceptable vehicle, wherein the anti-acne compound consists of 4% w/w hydroquinone, and 5% w/w ascorbic acid. In some embodiments, the anti-acne compound consists of 4% w/w hydroquinone, 5% w/w ascorbic acid, and 4% w/w kojic acid.

In some embodiments, the composition comprises azelaic acid and hydroquinone, or pharmaceutically acceptable salts thereof. In some embodiments, the composition consists of azelaic acid and hydroquinone, or pharmaceutically acceptable salts thereof. In some embodiments, the composition comprises two compounds comprising from 1% w/w to 10% w/w azelaic acid and from 0.1% w/w to 15% w/w hydroquinone. In some embodiments, the composition further comprises one or more of: resveratrol, hydrocortisone, dexpanthenol, kojic acid, and epigallocatechin gallate. In some embodiments, the composition comprises from 0.1% w/w to 3% w/w resveratrol. In some embodiments, the composition comprises from 0.5% w/w to 5% w/w hydrocortisone. In some embodiments, the composition comprises from 0.1% w/w to 3% w/w dexpanthenol. In some embodiments, the composition comprises from 2% w/w to 6% w/w kojic acid. In some embodiments, the composition comprises from 0.1% w/w to 6% w/w epigallocatechin gallate. In some embodiments, the composition comprises epigallocatechin gallate in the form of green tea extract.

In some embodiments, the composition comprises about 4% w/w hydroquinone, about 3% w/w azelaic acid, and about 2.5% w/w hydrocortisone. In some embodiments, the composition comprises about 4% w/w hydroquinone, about 3% w/w azelaic acid, about 2.5% w/w hydrocortisone, and about 4% w/w kojic acid.

In some embodiments, the composition comprises 4% w/w hydroquinone, 3% w/w azelaic acid, and 2.5% w/w hydrocortisone. In some embodiments, the composition comprises 4% w/w hydroquinone, 3% w/w azelaic acid, 2.5% w/w hydrocortisone, and 4% w/w kojic acid.

In some embodiments, the composition comprises an anti-acne compound and a pharmaceutically acceptable vehicle, wherein the anti-acne compound consists of about 4% w/w hydroquinone, about 3% w/w azelaic acid, and about 2.5% w/w hydrocortisone. In some embodiments, the anti-acne compound consists of about 4% w/w hydroquinone, about 3% w/w azelaic acid, about 2.5% w/w hydrocortisone, and about 4% w/w kojic acid.

In some embodiments, the composition comprises an anti-acne compound and a pharmaceutically acceptable vehicle, wherein the anti-acne compound consists of 4% w/w hydroquinone, 3% w/w azelaic acid, and 2.5% w/w hydrocortisone. In some embodiments, the anti-acne compound consists of 4% w/w hydroquinone, 3% w/w azelaic acid, 2.5% w/w hydrocortisone, and 4% w/w kojic acid.

In some embodiments, the composition comprises about 8% w/w hydroquinone, about 3% w/w azelaic acid, and about 2.5% w/w hydrocortisone. In some embodiments, the composition comprises about 8% w/w hydroquinone, about 3% w/w azelaic acid, about 2.5% w/w hydrocortisone, and about 4% w/w kojic acid.

In some embodiments, the composition comprises 8% w/w hydroquinone, 3% w/w azelaic acid, and 2.5% w/w hydrocortisone. In some embodiments, the composition comprises 8% w/w hydroquinone, 3% w/w azelaic acid, 2.5% w/w hydrocortisone, and 4% w/w kojic acid.

In some embodiments, the composition comprises an anti-acne compound and a pharmaceutically acceptable vehicle, wherein the anti-acne compound consists of about 8% w/w hydroquinone, about 3% w/w azelaic acid, and about 2.5% w/w hydrocortisone. In some embodiments, the anti-acne compound consists of about 8% w/w hydroquinone, about 3% w/w azelaic acid, about 2.5% w/w hydrocortisone, and about 4% w/w kojic acid.

In some embodiments, the composition comprises an anti-acne compound and a pharmaceutically acceptable vehicle, wherein the anti-acne compound consists of 8% w/w hydroquinone, 3% w/w azelaic acid, and 2.5% w/w hydrocortisone. In some embodiments, the anti-acne compound consists of 8% w/w hydroquinone, 3% w/w azelaic acid, 2.5% w/w hydrocortisone, and 4% w/w kojic acid.

In some embodiments, the composition comprises about 12% w/w hydroquinone, about 3% w/w azelaic acid, and about 2.5% w/w hydrocortisone. In some embodiments, the composition comprises about 12% w/w hydroquinone, about 3% w/w azelaic acid, about 2.5% w/w hydrocortisone, and about 4% w/w kojic acid.

In some embodiments, the composition comprises 12% w/w hydroquinone, 3% w/w azelaic acid, and 2.5% w/w hydrocortisone. In some embodiments, the composition comprises 12% w/w hydroquinone, 3% w/w azelaic acid, 2.5% w/w hydrocortisone, and 4% w/w kojic acid.

In some embodiments, the composition comprises an anti-acne compound and a pharmaceutically acceptable vehicle, wherein the anti-acne compound consists of about 12% w/w hydroquinone, about 3% w/w azelaic acid, and about 2.5% w/w hydrocortisone. In some embodiments, the anti-acne compound consists of about 12% w/w hydroquinone, about 3% w/w azelaic acid, about 2.5% w/w hydrocortisone, and about 4% w/w kojic acid.

In some embodiments, the composition comprises an anti-acne compound and a pharmaceutically acceptable vehicle, wherein the anti-acne compound consists of 12% w/w hydroquinone, 3% w/w azelaic acid, and 2.5% w/w hydrocortisone. In some embodiments, the anti-acne compound consists of 12% w/w hydroquinone, 3% w/w azelaic acid, 2.5% w/w hydrocortisone, and 4% w/w kojic acid.

In some embodiments, the composition comprises about 4% w/w hydroquinone, about 3% w/w azelaic acid, and about 1% w/w resveratrol. In some embodiments, the composition comprises about 4% w/w hydroquinone, about 3% w/w azelaic acid, about 1% w/w resveratrol, and about 4% w/w kojic acid.

In some embodiments, the composition comprises 4% w/w hydroquinone, 3% w/w azelaic acid, and 1% w/w resveratrol. In some embodiments, the composition comprises 4% w/w hydroquinone, 3% w/w azelaic acid, 1% w/w resveratrol, and 4% w/w kojic acid.

In some embodiments, the composition comprises an anti-acne compound and a pharmaceutically acceptable vehicle, wherein the anti-acne compound consists of about 4% w/w hydroquinone, about 3% w/w azelaic acid, and about 1% w/w resveratrol. In some embodiments, the anti-acne compound consists of about 4% w/w hydroquinone, about 3% w/w azelaic acid, about 1% w/w resveratrol, and about 4% w/w kojic acid.

In some embodiments, the composition comprises an anti-acne compound and a pharmaceutically acceptable vehicle, wherein the anti-acne compound consists of 4% w/w hydroquinone, 3% w/w azelaic acid, and 1% w/w resveratrol. In some embodiments, the anti-acne compound consists of 4% w/w hydroquinone, 3% w/w azelaic acid, 1% w/w resveratrol, and 4% w/w kojic acid.

In some embodiments, the composition comprises about 8% w/w hydroquinone, about 3% w/w azelaic acid, and about 1% w/w resveratrol. In some embodiments, the composition comprises about 8% w/w hydroquinone, about 3% w/w azelaic acid, about 1% w/w resveratrol, and about 4% w/w kojic acid.

In some embodiments, the composition comprises 8% w/w hydroquinone, 3% w/w azelaic acid, and 1% w/w resveratrol. In some embodiments, the composition comprises 8% w/w hydroquinone, 3% w/w azelaic acid, 1% w/w resveratrol, and 4% w/w kojic acid.

In some embodiments, the composition comprises an anti-acne compound and a pharmaceutically acceptable vehicle, wherein the anti-acne compound consists of about 8% w/w hydroquinone, about 3% w/w azelaic acid, and about 1% w/w resveratrol. In some embodiments, the anti-acne compound consists of about 8% w/w hydroquinone, about 3% w/w azelaic acid, about 1% w/w resveratrol, and about 4% w/w kojic acid.

In some embodiments, the composition comprises an anti-acne compound and a pharmaceutically acceptable vehicle, wherein the anti-acne compound consists of 8% w/w hydroquinone, 3% w/w azelaic acid, and 1% w/w resveratrol. In some embodiments, the anti-acne compound consists of 8% w/w hydroquinone, 3% w/w azelaic acid, 1% w/w resveratrol, and 4% w/w kojic acid.

In some embodiments, the composition comprises about 12% w/w hydroquinone, about 3% w/w azelaic acid, and about 1% w/w resveratrol. In some embodiments, the composition comprises about 12% w/w hydroquinone, about 3% w/w azelaic acid, about 1% w/w resveratrol, and about 4% w/w kojic acid.

In some embodiments, the composition comprises 12% w/w hydroquinone, 3% w/w azelaic acid, and 1% w/w resveratrol. In some embodiments, the composition comprises 12% w/w hydroquinone, 3% w/w azelaic acid, 1% w/w resveratrol, and 4% w/w kojic acid.

In some embodiments, the composition comprises an anti-acne compound and a pharmaceutically acceptable vehicle, wherein the anti-acne compound consists of about 12% w/w hydroquinone, about 3% w/w azelaic acid, and about 1% w/w resveratrol. In some embodiments, the anti-acne compound consists of about 12% w/w hydroquinone, about 3% w/w azelaic acid, about 1% w/w resveratrol, and about 4% w/w kojic acid.

In some embodiments, the composition comprises an anti-acne compound and a pharmaceutically acceptable vehicle, wherein the anti-acne compound consists of 12% w/w hydroquinone, 3% w/w azelaic acid, and 1% w/w resveratrol. In some embodiments, the anti-acne compound consists of 12% w/w hydroquinone, 3% w/w azelaic acid, 1% w/w resveratrol, and 4% w/w kojic acid.

In some embodiments, the composition comprises azelaic acid and resveratrol, or pharmaceutically acceptable salts thereof. In some embodiments, the composition consists of azelaic acid and resveratrol, or pharmaceutically acceptable salts thereof. In some embodiments, the composition comprises two compounds comprising from 1% w/w to 10% w/w azelaic acid and from 0.1% w/w to 3% w/w resveratrol. In some embodiments, the composition further comprises one or more of: kojic acid, ascorbic acid, and epigallocatechin gallate. In some embodiments, the composition comprises from 2% w/w to 10% w/w kojic acid. In some embodiments, the composition comprises from 1% w/w to 10% w/w ascorbic acid. In some embodiments, the composition comprises epigallocatechin gallate in the form of green tea extract.

In some embodiments, the composition comprises about 10% w/w azelaic acid and about 1% w/w resveratrol. In some embodiments, the composition comprises about 10% w/w azelaic acid, about 1% w/w resveratrol, and about 5% w/w ascorbic acid. In some embodiments, the composition comprises about 10% w/w azelaic acid, about 1% w/w resveratrol, about 4% w/w kojic acid, and about 2% w/w epigallocatechin gallate. In some embodiments, the composition comprises about 10% w/w azelaic acid, about 1% w/w resveratrol, about 5% w/w ascorbic acid, about 4% w/w kojic acid, and about 2% w/w epigallocatechin gallate.

In some embodiments, the composition comprises 10% w/w azelaic acid and 1% w/w resveratrol. In some embodiments, the composition comprises 10% w/w azelaic acid, 1% w/w resveratrol, and 5% w/w ascorbic acid. In some embodiments, the composition comprises 10% w/w azelaic acid, 1% w/w resveratrol, 4% w/w kojic acid, and 2% w/w epigallocatechin gallate. In some embodiments, the composition comprises 10% w/w azelaic acid, 1% w/w resveratrol, 5% w/w ascorbic acid, 4% w/w kojic acid, and 2% w/w epigallocatechin gallate.

In some embodiments, the composition comprises an anti-acne compound and a pharmaceutically acceptable vehicle, wherein the anti-acne compound consists of about 10% w/w azelaic acid and about 1% w/w resveratrol. In some embodiments, the anti-acne compound consists of about 10% w/w azelaic acid, about 1% w/w resveratrol, and about 5% w/w ascorbic acid. In some embodiments, the anti-acne compound consists of about 10% w/w azelaic acid, about 1% w/w resveratrol, about 4% w/w kojic acid, and about 2% w/w epigallocatechin gallate. In some embodiments, the anti-acne compound consists of about 10% w/w azelaic acid, about 1% w/w resveratrol, about 5% w/w ascorbic acid, about 4% w/w kojic acid, and about 2% w/w epigallocatechin gallate.

In some embodiments, the composition comprises an anti-acne compound and a pharmaceutically acceptable vehicle, wherein the anti-acne compound consists of 10% w/w azelaic acid and 1% w/w resveratrol. In some embodiments, the anti-acne compound consists of 10% w/w azelaic acid, 1% w/w resveratrol, and 5% w/w ascorbic acid. In some embodiments, the anti-acne compound consists of 10% w/w azelaic acid, 1% w/w resveratrol, 4% w/w kojic acid, and 2% w/w epigallocatechin gallate. In some embodiments, the anti-acne compound consists of 10% w/w azelaic acid, 1% w/w resveratrol, 5% w/w ascorbic acid, 4% w/w kojic acid, and 2% w/w epigallocatechin gallate.

In some embodiments, the composition comprises clindamycin and zinc pyrithione, or pharmaceutically acceptable salts thereof. In some embodiments, the composition consists of clindamycin and zinc pyrithione, or pharmaceutically acceptable salts thereof. In some embodiments, the composition comprises an anti-acne compound and a pharmaceutically acceptable vehicle, wherein the anti-acne compound consists of from 0.1% w/w to 5% w/w clindamycin and from 0.01% w/w to 1% w/w zinc pyrithione. In some embodiments, the composition comprises an anti-acne compound and a pharmaceutically acceptable vehicle, wherein the anti-acne compound consists of about 1% w/w clindamycin and about 0.25% w/w zinc pyrithione. In some embodiments, the composition comprises an anti-acne compound and a pharmaceutically acceptable vehicle, wherein the anti-acne compound consists of 1% w/w clindamycin and 0.25% w/w zinc pyrithione.

In some embodiments, the composition comprises azelaic acid and zinc pyrithione, or pharmaceutically acceptable salts thereof. In some embodiments, the composition consists of azelaic acid and zinc pyrithione, or pharmaceutically acceptable salts thereof. In some embodiments, the composition comprises an anti-acne compound and a pharmaceutically acceptable vehicle, wherein the anti-acne compound consists of from 1% w/w to 15% w/w azelaic acid and from 0.01% w/w to 1% w/w zinc pyrithione. In some embodiments, the composition comprises an anti-acne compound and a pharmaceutically acceptable vehicle, wherein the anti-acne compound consists of about 3% w/w azelaic acid and about 0.25% w/w zinc pyrithione. In some embodiments, the composition comprises an anti-acne compound and a pharmaceutically acceptable vehicle, wherein the anti-acne compound consists of about 6% w/w azelaic acid and about 0.25% w/w zinc pyrithione. In some embodiments, the composition comprises an anti-acne compound and a pharmaceutically acceptable vehicle, wherein the anti-acne compound consists of about 10% w/w azelaic acid and about 0.25% w/w zinc pyrithione. In some embodiments, the composition comprises an anti-acne compound and a pharmaceutically acceptable vehicle, wherein the anti-acne compound consists of 3% w/w azelaic acid and 0.25% w/w zinc pyrithione. In some embodiments, the composition comprises an anti-acne compound and a pharmaceutically acceptable vehicle, wherein the anti-acne compound consists of 6% w/w azelaic acid and 0.25% w/w zinc pyrithione. In some embodiments, the composition comprises an anti-acne compound and a pharmaceutically acceptable vehicle, wherein the anti-acne compound consists of 10% w/w azelaic acid and 0.25% w/w zinc pyrithione.

In some embodiments, the composition comprises clindamycin and azelaic acid, or pharmaceutically acceptable salts thereof. In some embodiments, the composition consists of clindamycin and azelaic acid, or pharmaceutically acceptable salts thereof. In some embodiments, the composition comprises an anti-acne compound and a pharmaceutically acceptable vehicle, wherein the anti-acne compound consists of from 0.1% w/w to 5% w/w clindamycin and from 0.1% w/w to 15% w/w azelaic acid. In some embodiments, the composition comprises an anti-acne compound and a pharmaceutically acceptable vehicle, wherein the anti-acne compound consists of about 1% w/w clindamycin and about 3% w/w azelaic acid. In some embodiments, the composition comprises an anti-acne compound and a pharmaceutically acceptable vehicle, wherein the anti-acne compound consists of about 1% w/w clindamycin and about 6% w/w azelaic acid. In some embodiments, the composition comprises an anti-acne compound and a pharmaceutically acceptable vehicle, wherein the anti-acne compound consists of about 1% w/w clindamycin and about 10% w/w azelaic acid. In some embodiments, the composition comprises an anti-acne compound and a pharmaceutically acceptable vehicle, wherein the anti-acne compound consists of 1% w/w clindamycin and 3% w/w azelaic acid. In some embodiments, the composition comprises an anti-acne compound and a pharmaceutically acceptable vehicle, wherein the anti-acne compound consists of 1% w/w clindamycin and 6% w/w azelaic acid. In some embodiments, the composition comprises an anti-acne compound and a pharmaceutically acceptable vehicle, wherein the anti-acne compound consists of 1% w/w clindamycin and 10% w/w azelaic acid.

In some embodiments, the composition comprises clindamycin, zinc pyrithione, azelaic acid, or pharmaceutically acceptable salts thereof. In some embodiments, the composition consists of clindamycin, zinc pyrithione, azelaic acid, or pharmaceutically acceptable salts thereof. In some embodiments, the composition comprises an anti-acne compound and a pharmaceutically acceptable vehicle, wherein the anti-acne compound consists of from 0.1% w/w to 5% w/w clindamycin, from 0.01% w/w to 1% w/w zinc pyrithione, and from 0.1% w/w to 15% w/w azelaic acid. In some embodiments, the composition comprises an anti-acne compound and a pharmaceutically acceptable vehicle, wherein the anti-acne compound consists of about 1% w/w clindamycin, about 0.25% w/w zinc pyrithione, and about 2% w/w azelaic acid. In some embodiments, the composition comprises an anti-acne compound and a pharmaceutically acceptable vehicle, wherein the anti-acne compound consists of 1% w/w clindamycin, 0.25% w/w zinc pyrithione, and 2% w/w azelaic acid.

In some embodiments, the composition comprises hydroquinone and desonide, or pharmaceutically acceptable salts thereof. In some embodiments, the composition consists of hydroquinone and desonide, or pharmaceutically acceptable salts thereof. In some embodiments, the composition comprises from 0.1% w/w to 15% w/w hydroquinone and from 0.005% w/w to 0.5% w/w desonide. In some embodiments, the composition comprises about 4% w/w hydroquinone and about 0.05% w/w desonide. In some embodiments, the composition comprises 4% w/w hydroquinone and 0.05% w/w desonide. In some embodiments, the composition comprises an anti-acne compound and a pharmaceutically acceptable vehicle, wherein the anti-acne compound consists of about 4% w/w hydroquinone and about 0.05% w/w desonide. In some embodiments, the composition comprises an anti-acne compound and a pharmaceutically acceptable vehicle, wherein the anti-acne compound consists of 4% w/w hydroquinone and 0.05% w/w desonide.

In some embodiments, the composition comprises metronidazole and niacinamide, or pharmaceutically acceptable salts thereof. In some embodiments, the composition consists of metronidazole and niacinamide, or pharmaceutically acceptable salts thereof. In some embodiments, the composition comprises from 0.1% w/w to 3% w/w metronidazole and from 1% w/w to 10% w/w niacinamide. In some embodiments, the composition further comprises azelaic acid. In some embodiments, the azelaic acid is present in an amount from 0.5% w/w to 20% w/w. In some embodiments, the composition comprises about 1% w/w metronidazole, about 4% w/w niacinamide, and about 2% w/w azelaic acid. In some embodiments, the composition comprises 1% w/w metronidazole, 4% w/w niacinamide, and about 2% w/w azelaic acid. In some embodiments, the composition comprises an anti-acne compound and a pharmaceutically acceptable vehicle, wherein the anti-acne compound consists of about 1% w/w metronidazole, about 4% w/w niacinamide, and about 2% w/w azelaic acid. In some embodiments, the composition comprises an anti-acne compound and a pharmaceutically acceptable vehicle, wherein the anti-acne compound consists of 1% w/w metronidazole, 4% w/w niacinamide, and 2% w/w azelaic acid.

In some embodiments, the composition comprises clindamycin, niacinamide, azelaic acid, or pharmaceutically acceptable salts thereof. In some embodiments, the composition consists of clindamycin, niacinamide, azelaic acid, or pharmaceutically acceptable salts thereof. In some embodiments, the composition comprises from 0.1% w/w to 3% w/w clindamycin, from 1% w/w to 10% w/w niacinamide, and from 0.5% w/w to 20% w/w azelaic acid. In some embodiments, the composition comprises about 1% w/w clindamycin, about 4% w/w niacinamide, and about 2% w/w azelaic acid. In some embodiments, the composition comprises an anti-acne compound and a pharmaceutically acceptable vehicle, wherein the anti-acne compound consists of about 1% w/w clindamycin, about 4% w/w niacinamide, and about 2% w/w azelaic acid. In some embodiments, the composition comprises an anti-acne compound and a pharmaceutically acceptable vehicle, wherein the anti-acne compound consists of 1% w/w clindamycin, 4% w/w niacinamide, and 2% w/w azelaic acid.

In some embodiments, the composition comprises azelaic acid and ascorbic acid, or pharmaceutically acceptable salts thereof. In some embodiments, the composition consists of azelaic acid and ascorbic acid, or pharmaceutically acceptable salts thereof. In some embodiments, the composition comprises from 0.5% w/w to 20% w/w azelaic acid and from 1% w/w to 10% w/w ascorbic acid. In some embodiments, the composition further comprises kojic acid. In some embodiments, the kojic acid is present in an amount from 2% w/w to 6% w/w. In some embodiments, the composition comprises about 10% w/w azelaic acid and about 5% w/w ascorbic acid. In some embodiments, the composition comprises about 10% w/w azelaic acid, about 5% w/w ascorbic acid, and about 4% w/w kojic acid. In some embodiments, the composition comprises 10% w/w azelaic acid and 5% w/w ascorbic acid. In some embodiments, the composition comprises 10% w/w azelaic acid, 5% w/w ascorbic acid, and 4% w/w kojic acid. In some embodiments, the composition comprises about 10% w/w azelaic acid and about 5% w/w ascorbic acid. In some embodiments, the composition comprises an anti-acne compound and a pharmaceutically acceptable vehicle, wherein the anti-acne compound consists of about 10% w/w azelaic acid, about 5% w/w ascorbic acid, and about 4% w/w kojic acid. In some embodiments, the composition comprises an anti-acne compound and a pharmaceutically acceptable vehicle, wherein the anti-acne compound consists of 10% w/w azelaic acid, 5% w/w ascorbic acid, and 4% w/w kojic acid.

In some embodiments, the composition comprises tretinoin, clindamycin, and azelaic acid, or pharmaceutically acceptable salts thereof. In some embodiments, the composition consists of tretinoin, clindamycin, and azelaic acid, or pharmaceutically acceptable salts thereof. In some embodiments, the composition comprises about 0.01% w/w tretinoin, about 1% w/w clindamycin, and about 2% w/w azelaic acid. In some embodiments, the composition comprises about 0.01% w/w tretinoin, about 1% w/w clindamycin, and about 3% w/w azelaic acid. In some embodiments, the composition comprises about 0.01% w/w tretinoin, about 1% w/w clindamycin, and about 4% w/w azelaic acid. In some embodiments, the composition comprises 0.01% w/w tretinoin, 1% w/w clindamycin, and 3% w/w azelaic acid. In some embodiments, the composition comprises 0.01% w/w tretinoin, 1% w/w clindamycin, and 4% w/w azelaic acid. In some embodiments, the composition comprises about 0.003% w/w tretinoin, about 1% w/w clindamycin, and about 2% w/w azelaic acid. In some embodiments, the composition comprises about 0.005% w/w tretinoin, about 1% w/w clindamycin, and about 2% w/w azelaic acid. In some embodiments, the composition comprises 0.005% w/w tretinoin, 1% w/w clindamycin, and 2% w/w azelaic acid. In some embodiments, the composition comprises about 0.007% w/w tretinoin, about 1% w/w clindamycin, and about 2% w/w azelaic acid. In some embodiments, the composition comprises 0.007% w/w tretinoin, 1% w/w clindamycin, and 2% w/w azelaic acid.

In some embodiments, the composition comprises 0.01% w/w tretinoin, 1% w/w clindamycin, and 2% w/w azelaic acid. In some embodiments, the composition comprises 0.003% w/w tretinoin, 1% w/w clindamycin, and 2% w/w azelaic acid. In some embodiments, the composition comprises 0.005% w/w tretinoin, 1% w/w clindamycin, and 2% w/w azelaic acid. In some embodiments, the composition comprises 0.007% w/w tretinoin, 1% w/w clindamycin, and 2% w/w azelaic acid.

In some embodiments, the composition comprises an anti-acne compound and a pharmaceutically acceptable vehicle, wherein the anti-acne compound consists of about 0.01% w/w tretinoin, about 1% w/w clindamycin, and about 2% w/w azelaic acid. In some embodiments, the anti-acne compound consists of about 0.003% w/w tretinoin, about 1% w/w clindamycin, and about 2% w/w azelaic acid. In some embodiments, the anti-acne compound consists of about 0.005% w/w tretinoin, about 1% w/w clindamycin, and about 2% w/w azelaic acid. In some embodiments, the anti-acne compound consists of about 0.007% w/w tretinoin, about 1% w/w clindamycin, and about 2% w/w azelaic acid.

In some embodiments, the composition comprises an anti-acne compound and a pharmaceutically acceptable vehicle, wherein the anti-acne compound consists of 0.01% w/w tretinoin, 1% w/w clindamycin, and 2% w/w azelaic acid. In some embodiments, the anti-acne compound consists of 0.003% w/w tretinoin, 1% w/w clindamycin, and 2% w/w azelaic acid. In some embodiments, the anti-acne compound consists of 0.005% w/w tretinoin, 1% w/w clindamycin, and 2% w/w azelaic acid. In some embodiments, the anti-acne compound consists of 0.007% w/w tretinoin, 1% w/w clindamycin, and 2% w/w azelaic acid.

In certain embodiments, the composition further comprises a pharmaceutically acceptable vehicle and/or one or more inactive ingredients. At least one inactive ingredient can be a pharmaceutically acceptable vehicle. In some embodiments, at least one inactive ingredient is a preservative. In some embodiments, the composition further comprises at least one pharmaceutically acceptable vehicle, at least one emulsifier, at least one glycol, and at least one preservative. In certain embodiments, the composition further comprises one or more of the following inactive ingredients: water, vegetable glycerin, stearic acid, myristyl myristate, cetearyl alcohol, ceteareth-20, glyceryl stearate, jojoba seed oil, soybean oil, cetyl alcohol, carbomer, shea butter, calendula flower oil, passion fruit seed oil, rice bran oil, acai palm fruit oil, phenoxyethanol, and/or ethylhexylglycerin. In one embodiment, the composition further comprises the following inactive ingredients water, aloe barbadensis leaf juice, C13-14 isoparaffin, caprylic/capric triglyceride, laureth-7, maltodextrin, phenoxyethanol, polyacrylamide, tocopheryl acetate, and triethylene glycol.

In some embodiments, the three anti-acne or anti-photoaging compounds are active at the same pH or in the same pH range. In other embodiments, the pH of the composition ranges, e.g., from about 3.0 to about 7.0. In some embodiments, the pH of the composition ranges, e.g., from about 3.5 to about 6.0. In some embodiments, the pH of the composition ranges, e.g., from about 4.0 to about 5.0.

The compositions according to the disclosure can be made as follows:

In some embodiments, the composition batch size ranges, e.g., from about 5 g to about 100 kg. In some embodiments, the composition batch size ranges, e.g., from about 100 g to about 10 kg. In some embodiments, the composition batch size ranges, e.g., from about 0.5 kg to about 3 kg. In certain embodiments, the composition batch is divided into 30 g aliquots.

In some embodiments, the composition batch size can range, e.g., from about 5 mL to about 100 L, from about 100 mL to 10 L, or from about 0.5 L to 3 L. In certain embodiments, the composition batch is divided into 30 mL aliquots.

### C. Methods of Treatment

In another aspect, the disclosure provides a method for the treatment of a skin disorder such as, but not limited to, acne, photoaging, wrinkles and/or uneven pigmentation in a subject in need thereof, comprising administering to the skin of the subject a composition according to the disclosure.

In some embodiments, the composition is administered topically. The topical compositions of the present disclosure can be provided in the form of a cream (ointment), a gel, or lotion. The pharmaceutically acceptable vehicle is selected according to the desired final form of the topical composition (cream or ointment, gel, lotion, and the like) from the types of vehicles known in the art for topical application of active ingredients.

According to some embodiments, administration of the topical pharmaceutical composition to the skin of a subject results in reduction of fine lines and wrinkles, reduction in acne, skin firming, improvement in skin texture, improvement in the skin's elasticity, improvement in skin luminosity, reduction of uneven pigmentation, skin hydration, skin moisturization, reduction in skin dehydration, and improvement of even skin tone.

In some embodiments, the frequency of application of the disclosed compositions to the skin of a subject is determined by a medical provider. In some embodiments, the frequency of application of the disclosed compositions to the skin of a subject may be one, two, or three times per day. In certain embodiments, the frequency of application is once per day. In some embodiments the application of the disclosed compositions to the skin of a subject occurs at night. In certain embodiments, the application of the disclosed compositions occurs before the subject goes to sleep.

According to some embodiments, the method includes evaluating the skin of a subject, such as, but not limited to, by using telemedicine. According to some embodiments, a first topical pharmaceutical composition is administered to the subject. The skin of the treated subject may then be reevaluated. According to some embodiments, if the skin evaluated has not improved, a second topical pharmaceutical composition is administered. The second topical pharmaceutical composition may comprise ingredients that cause enhanced skin irritation when compared to the first topical pharmaceutical composition.

In some embodiments, the treated skin of the subject is then evaluated for skin brightness, discoloration, fine lines, and/or wrinkles. In certain embodiments, evaluating the skin of the subject includes one or more of the following: creation and use of a portal (e.g., through the internet), which allows secure examination of high-resolution photographs; remote examination of high-resolution photographs; and in person examination by a qualified grader. In other embodiments, the skin of the subject is evaluated via a form of telemedicine via secure uploading of the subject's medical history and digital high-resolution photographs online or through the internet. In still other embodiments, evaluating the skin of the subject includes evaluating skin by profilometry; evaluating skin tone; evaluating skin color; evaluating skin firmness; evaluating skin elasticity; evaluating skin hydration; and evaluating skin aging by visual assessments. In some embodiments, evaluating the skin of the subject includes one or more of the following: profilometric analysis; measurements with a CUTOMETER® MPA 580; measurements with a CHROMAMETER CR300®; measurements with a CORNEOMETER® CM825; expert visual assessments; and visual assessments with Visia-CR® Capture.

In some embodiments, the expected effects of the treatment including, but non-limited to, visible reduction of fine lines and wrinkles, skin firming, improvement in skin texture, improvement in the skin's elasticity, improvement in skin luminosity, reduction of uneven pigmentation, hydrating, moisturizing, combating skin dehydration, and/or encouraging even skin tone, are evaluated after an interval of time.

For example, improvement of acne, e.g., less acne, or of photoaging, e.g., less discoloration, fine lines, and/or wrinkles, is evaluated after an interval of time. The interval of time can range, e.g., from about 1 day to about a year, or from about 1 week to about 6 months (e.g., about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks). In one embodiment, the first interval is four weeks. In other embodiments, evaluating the skin of the subject occurs at one or more intervals of time over the course of treatment. In some embodiments, the method of treatment of acne and photoaging in a subject in need thereof is applied over a period of time that can range, e.g., from about 1 day to about 50 years, or from about 1 week to about 25 years (e.g., about 3 months, about 6 months, about 1 year, about 5 years, or about 10 years).

In other embodiments, the acne is caused by P. acnes, and the antimicrobial may target one or more of P. acnes, Staphylococcus aureus, and Staphylococcus epidermis. In other embodiments, the acne is caused by Demodex folliculorum, and metronidazole is used to target Demodex folliculorum. In some embodiments, the acne to be treated is non-inflammatory acne, also known as comedones, (e.g., blackheads and white heads). In other embodiments, the acne is inflammatory acne (e.g., papules, pustules, nodules, and cysts). In still other embodiments, the acne is a combination of non-inflammatory acne and inflammatory acne.

In certain embodiments, the acne may be classified by its severity. When a subject has several comedones but very few papules and pustules, then the subject has mild acne. If a subject has a mix of comedones and several inflamed papules and pustules existing together, the acne is mild to moderate acne. If a subject also has some nodules along with papules and pustules, the acne is moderate acne. Deep cysts or any type of acne that leaves behind permanent pitted or saucer-shaped scars is categorized as severe acne. In certain embodiments, evaluating the skin of the subject includes evaluating the severity of the acne.

A physician, clinician, or scientist having ordinary skill in the art can readily determine and prescribe the effective amount of the pharmaceutical composition required. For example, the physician, clinician, or scientist could start doses of the pharmaceutical compounds of the disclosure at levels lower than that required in order to achieve the desired therapeutic effect and gradually increase the dosage until the desired effect is achieved.

It will be understood by those having ordinary skill in the art that the specific dose level and frequency of dosage for any particular patient or subject may be varied and will depend upon a variety of factors, including the activity of the specific composition employed, the metabolic stability and length of action of that composition, the age, body weight, general health, gender, diet, and the severity of the particular dermatological condition being treated. In addition, specific dose level and frequency of dosage for any particular patient also may depend on factors including, but not limited to, skin sensitivity, other medications, acne type, allergies, and prior experiences with dermatologic treatments. For example, some patients may be treated using methods of this disclosure over a period of years if the acne and/or photoaging is a chronic condition.

### D. Kits

In additional aspects, pharmaceutical kits are provided. The kit includes a sealed container approved for the storage of pharmaceutical compositions and containing one of the above-described pharmaceutical compositions. In some embodiments, the sealed container minimizes the contact of air with the ingredients, e.g., an airless bottle. In other embodiments, the sealed container is a sealed tube. In certain embodiments, the sealed container is not a pump bottle. An instruction for the use of the composition and the information about the composition are to be included in the kit.

The following examples are provided to further elucidate the advantages and features of the present application, but are not intended to limit the scope of the application. The examples are for the illustrative purposes only. USP pharmaceutical grade products were used in preparing the formulations described below.

### EXAMPLES

### Example 1. Evaluating the Skin by Profilometry

The skin of the subjects with acne and/or photoaging treated with pharmaceutical compositions of the disclosure are evaluated based on profilometry. Subjects are positioned on their backs with their head in line with the midline of their body. They are asked to close their eyes and maintain a neutral expression. Test sites on the skin of the subject are located using replica locating rings ensuring that each ring lay flat on the skin. The skin is not stretched or pulled during ring placement. Each ring is placed on the left and right periorbital areas of the face with the tab directed toward the back of the head. The foam and paper portions of each ring are aligned. Subjects are asked to turn their head so that the side of the head being evaluated is as horizontal as possible. The transparency film is then placed over the subject's face. Full locating rings, with centers, are then placed onto the film exactly over the site which had been selected. Landmarks are then traced onto the film using an indelible marker pen. The film is then removed from the face and labeled to identify the subject. The film is stored in a cool, dry location until next use.

### Replica Generation

The subject is placed in an identical manner to that described above, and landmarks on the transparency film are lined up with the subject's facial features. A skin marker pen is then used to make dots through the film onto the face of the subject to enable exact location of the test sites. The ring is then positioned on the face, and the replicas are generated by filling the well in the center of the ring with SILFLO® (JS Davis, Hert) material. Once the replica has set completely (approximately 5 minutes), it is removed from the skin, allowed to dry skin side up for a few minutes, and then placed in a storage sleeve.

### Profilometric Analysis

The following equipment and software are used: PC: IBM® compatible Pentium® III 500 MHz with 256 MB memory running under Windows® 2000 Professional. Video: COHUR solid state B&W camera, 50 mm lens/30 mm extension, CORECOR TCI; Ultra frame grabber. OPTIMAS® v6.5, Microsoft® EXCEL 2000, STATSOFT® STATISTICA 6. A collimated light source directed at a 25° angle from the plane of the replica is used.

The replica is placed in a holder that fixed the direction of the tab position of the replica so that the replica could be rotated to align the tab direction normal or parallel to the incident light direction. The replicas are taken from the crow's feet area adjacent to each eye with the tab direction pointing toward the ear. The NORMAL sampling orientation provides texture measurements sensitive to the MAJOR, expression-induced lines (crow's feet). The PARALLEL sampling orientation provides texture measurements sensitive to the MINOR, fine lines. The general background gradient of light intensity is adjusted by applying a 1st order correction in the direction of the light propagation.

The shadow texture produced by the oblique lighting of the negative replica is analyzed by two types of assay methods (A and B):

### Method A

The luminance is measured along a set of 10 equal length parallel lines (passes) running across the replica parallel to the lighting direction. The variations in luminance are treated as indicative of the roughness and analyzed by the following traditional surface roughness statistics:
Rz—the average maximum difference in luminance value for five equal length segments in each of the 10 lines traversing the sample;
Ra—the average deviation of the luminance curve about the mean luminance for the same 10 lines;
The “R” parameters are reported in the units of brightness (Grey Levels) ranging from 0 to 255;
FSpace—distance between markers placed on the lines at luminance changes indicative of fine lines; and
FNum—number markers per mm placed on the lines at luminance changes indicative of fine lines.

### Method B

The replica image area is then divided into 10 equal width bands or sub-areas. The shadow-like features are detected in each of these bands according to their luminance values being less than the detection threshold. The following four parameters are determined from the detected features:
Spacing—the mean distance in millimeters between adjacent detected features (i.e., spacing between the midpoints of adjacent shadowy features);
Breadth—the average breadth in millimeters of the detected features in millimeters. This parameter is proportional to the depth of the wrinkle producing the shadow; Shadows-percent of the sampled replica area with luminance values less than the detection threshold. This is the relative area of shadows cast by the wrinkles and fine lines in the replica; and
NumWr—the total number of features detected in the 10 bands or sub-areas used to calculate spacing and breadth.

### Example 2. Evaluating the Skin Firmness and Elasticity

The skin of the subjects with acne and/or photoaging treated with pharmaceutical compositions of the disclosure are evaluated based on measurements to study any changes in the viscoelastic properties of the skin as a result of treatment with the compositions of the disclosure. The measurements are performed using the CUTOMETER® MPA 580 (Courage and Khazaka, Germany). The measuring principle is based on the suction method. Negative pressure is created in the device, and the skin is drawn into the aperture of the probe. Inside the probe, the penetration depth is determined by a noncontact optical measuring system. This optical measuring system consists of a light source and a light receptor, as well as two prisms facing each other, which project the light from transmitter to receptor. The light intensity varies due to the penetration depth of the skin. The resistance of the skin to be sucked up by the negative pressure (firmness) and its ability to return into its original position (elasticity) are displayed as curves at the end of each measurement using MICROSOFT WINDOWS® based software.

### Example 3. Evaluating the Skin Tone and Color

The skin of the subjects with acne and/or photoaging treated with pharmaceutical compositions of the disclosure are evaluated for skin tone and color changes. Evaluation is performed using a CHROMAMETER CR300® (Courage and Khazaka, Germany) in person. The measuring head of the CR-300 uses diffuse illumination/0° viewing geometry. A pulsed xenon arc (PXA) lamp inside a mixing chamber provides diffuse, uniform lighting over the 8 mm-diameter specimen area. Only the light reflected perpendicular to the specimen surface is collected by the optical-fiber cable for color analysis. This instrument measures the amount of light reflected from the skin and quantifies this into a numerical value using the L*a*b* color scale, where L*(100) equates to total white and L*(0) equates to total black. Therefore, the L* value is inversely proportional to the Fitzpatrick visual scale of skin tone. The instrument is allowed to warm up for 30 minutes prior to use.

### Example 4. Evaluating the Skin Hydration

The skin of the subjects with acne and/or photoaging treated with pharmaceutical compositions of the disclosure are evaluated for humectant properties, performed using the CORNEOMETER® CM825 (Courage and Khazaka, Germany). This instrument relies on the dielectric constant, a physical property of water, which is relatively high and as such will affect the capacitance of a capacitor. Any change in the dielectric constant due to skin moisture variations will alter the capacitance of the precision capacitor in the instrument. These variations are detected electronically and are converted into a value by the CORNEOMETER®. A 15 minute warm-up period is allowed before using the CORNEOMETER®.

Prior to assessment, subjects must have been in the controlled environment (at a temperature of 22° C.±2° C. and at a relative humidity of 45%±5%) for at least 30 minutes.

Three measurements are made using the probe attachment of the CORNEOMETER® at each of the test sites on the skin of the subject, between each assessment the probe attachment of the CORNEOMETER® is pressed onto a dry tissue. The next assessment is not performed until a value of 5 or less is displayed by the instrument.

### Example 5. Evaluating the Skin by Expert Visual Assessment

The skin of the subjects with acne and/or photoaging treated with pharmaceutical compositions of the disclosure is evaluated by the same qualified grader at each time-point for the duration of the study according to the Glogau Classification of Aging scale. Table 1 shows the skin characteristics associated with each respective classification on the Glogau Classification of Aging scale.

TABLE 1
The Glogau Classification of Aging Scale

| | | Typical | | |
|---|---|---|---|---|
| Group | Classification | Age | Description | Skin Characteristics |
| I | Mild | 28-35 | No wrinkles | Early Photoaging: |
| | | | | Mild pigment changes, |
| | | | | no keratosis, minimal |
| | | | | wrinkles, minimal |
| | | | | or no makeup |
| II | Moderate | 35-50 | Wrinkles in | Early to Moderate |
| | | | motion | Photoaging: Early |
| | | | | brown spots visible, |
| | | | | keratosis palpable |
| | | | | but not visible, |
| | | | | parallel smile lines |
| | | | | begin to appear, |
| | | | | wears some foundation |
| III | Advanced | 50-65 | Wrinkles at | Advanced Photoaging: |
| | | | rest | Obvious |
| | | | | discolorations, |
| | | | | visible capillaries |
| | | | | (telangiectasias), |
| | | | | visible keratosis, |
| | | | | wears heavier |
| | | | | foundation always |
| IV | Severe | 60-75 | Only | Severe Photoaging: |
| | | | wrinkles | Yellow-gray skin |
| | | | | color, prior skin |
| | | | | malignancies, |
| | | | | wrinkles |
| | | | | throughout-no |
| | | | | normal skin, |
| | | | | cannot wear makeup |
| | | | | because it cakes |
| | | | | and cracks |

Illumination of the test sites on the skin of the subject is by a 60 watt pearl bulb placed approximately 30 cm from the test site on the skin of the subject.

### Example 6. Evaluating the Skin by Visual Assessment with Visia-CR® Capture

The skin of the subjects with acne and/or photoaging treated with pharmaceutical compositions of the disclosure are photographed and evaluated by the same qualified grader at each time-point for the duration of the study according to the Glogau Classification of Aging scale (Table 1, supra).

The Visia-CR® captures multiple lighting modalities in one computer-controlled sequence. Subjects can be photographed using standard light, UV, cross-polarization, and parallel polarization techniques. The Visia-CR® is used to capture one full-face, and two side-view images (one left side and one right side), high-resolution digital image of each subject with their eyes closed. Subjects are instructed to remain in a relaxed state while photos are captured using the Visia-CR® equipment.

### EQUIVALENTS

Although the invention has been described with reference to the above examples, it will be understood that modifications and variations are encompassed within the spirit and scope of the invention.

1. A composition comprising:
from 1% w/w to 10% w/w ascorbic acid and
from 1% w/w to 15% w/w hydroquinone.

2. A composition comprising:
from 1% w/w to 10% w/w azelaic acid and
from 0.1% w/w to 15% w/w hydroquinone.

3. The composition of claim 1 or 2, further comprising one or more of: resveratrol, hydrocortisone, dexpanthenol, kojic acid, and epigallocatechin gallate.

4. The composition of claim 3, wherein the resveratrol is present in an amount from 0.1% w/w to 3% w/w.

5. The composition of claim 3 or 4, wherein the hydrocortisone is present in an amount from 0.5% w/w to 5% w/w.

6. The composition of any one of claims 3-5, wherein the dexpanthenol is present in an amount from 0.1% w/w to 3% w/w.

7. The composition of any one of claims 3-6, wherein the kojic acid is present in an amount from 2% w/w to 6% w/w.

8. The composition of any one of claims 3-7, wherein the epigallocatechin gallate is present in an amount from 0.1% w/w to 6% w/w.

9. A composition comprising:
from 1% w/w to 10% w/w azelaic acid and
from 0.1% w/w to 3% w/w resveratrol.

10. The composition of claim 9, further comprising one or more of: kojic acid, ascorbic acid, and epigallocatechin gallate.

11. The composition of claim 10, wherein the kojic acid is present in an amount from 2% w/w to 10% w/w.

12. The composition of claim 10 or 11, wherein the ascorbic acid is present in an amount from 1% w/w to 10% w/w.

13. The composition of any one of claim 3-8 or 10-12, wherein the epigallocatechin gallate is present in the form of green tea extract.

14. The composition of claim 2, comprising:
about 4% w/w hydroquinone,
about 3% w/w azelaic acid, and
about 2.5% w/w hydrocortisone.

15. The composition of claim 1, comprising:
about 4% w/w hydroquinone,
about 2.5% w/w hydrocortisone, and
about 5% w/w ascorbic acid.

16. The composition of claim 14 or 15, further comprising about 4% w/w kojic acid.

17. The composition of claim 2, comprising:
about 8% w/w hydroquinone,
about 3% w/w azelaic acid, and
about 2.5% w/w hydrocortisone.

18. The composition of claim 1, comprising:
about 8% w/w hydroquinone,
about 2.5% w/w hydrocortisone, and
about 5% w/w ascorbic acid.

19. The composition of claim 17 or 18, further comprising about 4% w/w kojic acid.

20. The composition of claim 2, comprising:
about 12% w/w hydroquinone,
about 3% w/w azelaic acid, and
about 2.5% w/w hydrocortisone.

21. The composition of claim 1, comprising:
about 12% w/w hydroquinone,
about 2.5% w/w hydrocortisone, and
about 5% w/w ascorbic acid.

22. The composition of claim 20 or 21, further comprising about 4% w/w kojic acid.

23. The composition of claim 2, comprising:
about 4% w/w hydroquinone,
about 3% w/w azelaic acid, and
about 1% w/w resveratrol.

24. The composition of claim 1, comprising:
about 4% w/w hydroquinone,
about 1% w/w dexpanthenol, and
about 5% w/w ascorbic acid.

25. The composition of claim 23 or 24, further comprising about 4% w/w kojic acid.

26. The composition of claim 2, comprising:
about 8% w/w hydroquinone,
about 3% w/w azelaic acid, and
about 1% w/w resveratrol.

27. The composition of claim 1, comprising:
about 8% w/w hydroquinone,
about 5% w/w ascorbic acid, and
about 1% w/w dexpanthenol.

28. The composition of claim 26 or 27, further comprising about 4% w/w kojic acid.

29. The composition of claim 2, comprising:
about 12% w/w hydroquinone,
about 3% w/w azelaic acid, and
about 1% w/w resveratrol.

30. The composition of claim 1, comprising:
about 12% w/w hydroquinone,
about 5% w/w ascorbic acid, and
about 1% w/w dexpanthenol.

31. The composition of claim 29 or 30, further comprising about 4% w/w kojic acid.

32. The composition of claim 1, comprising:
about 4% w/w hydroquinone and
about 5% w/w ascorbic acid.

33. The composition of claim 32, further comprising about 4% w/w kojic acid.

34. The composition of claim 9, comprising:
about 10% w/w azelaic acid and
about 1% w/w resveratrol.

35. The composition of claim 9, comprising:
about 10% w/w azelaic acid,
about 1% w/w resveratrol, and
about 5% w/w ascorbic acid.

36. The composition of claim 34 or 35, further comprising one or both of:
about 4% w/w kojic acid and
about 2% w/w epigallocatechin gallate.

37. A composition comprising an anti-acne compound and a pharmaceutically acceptable vehicle, wherein the anti-acne compound consists of:
from 0.1% w/w to 5% w/w clindamycin and
from 0.01% w/w to 1% w/w zinc pyrithione.

38. The composition of claim 37, wherein the anti-acne compound consists of:
about 1% w/w clindamycin and
about 0.25% w/w zinc pyrithione.

39. A composition comprising an anti-acne compound and a pharmaceutically acceptable vehicle, wherein the anti-acne compound consists of:
from 1% w/w to 15% w/w azelaic acid and
from 0.01% w/w to 1% w/w zinc pyrithione.

40. The composition of claim 39, wherein the anti-acne compound consists of:
about 3% w/w azelaic acid and
about 0.25% w/w zinc pyrithione.

41. The composition of claim 39, wherein the anti-acne compound consists of:
about 6% w/w azelaic acid and
about 0.25% w/w zinc pyrithione.

42. The composition of claim 39, wherein the anti-acne compound consists of:
about 10% w/w azelaic acid and
about 0.25% w/w zinc pyrithione.

43. A composition comprising, an anti-acne compound and a pharmaceutically acceptable vehicle, wherein the anti-acne compound consists of:
from 0.1% w/w to 5% w/w clindamycin and
from 0.1% w/w to 15% w/w azelaic acid.

44. The composition of claim 43, wherein the anti-acne compound consists of:
about 1% w/w clindamycin and
about 3% w/w azelaic acid.

45. The composition of claim 43, wherein the anti-acne compound consists of:
about 1% w/w clindamycin and
about 6% w/w azelaic acid.

46. The composition of claim 43, wherein the anti-acne compound consists of:
about 1% w/w clindamycin and
about 10% w/w azelaic acid.

47. A composition comprising, an anti-acne compound and a pharmaceutically acceptable vehicle, wherein the anti-acne compound consists of:
from 0.1% w/w to 5% w/w clindamycin,
from 0.1% w/w to 15% w/w azelaic acid, and
from 0.01% w/w to 1% w/w zinc pyrithione.

48. The composition of claim 47, wherein the anti-acne compound consists of:
about 1% w/w clindamycin,
about 2% w/w azelaic acid, and
about 0.25% w/w zinc pyrithione.

49. A composition comprising, an anti-acne compound and a pharmaceutically acceptable vehicle, wherein the anti-acne compound consists of:
from 0.1% w/w to 5% w/w clindamycin,
from 0.1% w/w to 15% w/w azelaic acid, and
from 1% w/w to 10% w/w niacinamide.

50. The composition of claim 47, wherein the anti-acne compound consists of:
about 1% w/w clindamycin,
about 2% w/w azelaic acid, and
about 4% w/w niacinamide.

51. A composition comprising:
from 0.1% w/w to 15% w/w hydroquinone and
from 0.005% w/w to 0.5% w/w desonide.

52. The composition of claim 51, comprising:
about 4% w/w hydroquinone and
about 0.05% w/w desonide.

53. A composition comprising:
from 0.1% w/w to 3% w/w metronidazole and
from 1% w/w to 10% w/w niacinamide.

54. The composition of claim 53, further comprising azelaic acid.

55. The composition of claim 54, wherein the azelaic acid is present in an amount from 0.5% w/w to 20% w/w.

56. The composition of any one of claims 53-55, comprising:
about 1% w/w metronidazole,
about 4% w/w niacinamide, and
about 2% w/w azelaic acid.

57. A composition comprising:
from 0.5% w/w to 20% w/w azelaic acid and
from 1% w/w to 10% w/w ascorbic acid.

58. The composition of claim 57, further comprising kojic acid.

59. The composition of claim 58, wherein the kojic acid is present in an amount of 2% to 6% w/w.

60. The composition of any one of claims 57-59, comprising:
about 10% w/w azelaic acid and
about 5% w/w ascorbic acid.

61. The composition of any one of claims 57-59, further comprising about 4% w/w kojic acid.

62. A composition comprising:
about 0.01% w/w tretinoin,
about 1% w/w clindamycin, and
about 2% w/w azelaic acid.

63. A composition comprising:
about 0.003% w/w tretinoin,
about 1% w/w clindamycin, and
about 2% w/w azelaic acid.

64. A composition comprising:
about 0.005% w/w tretinoin,
about 1% w/w clindamycin, and
about 2% w/w azelaic acid.

65. A composition comprising:
about 0.007% w/w tretinoin,
about 1% w/w clindamycin, and
about 2% w/w azelaic acid.

66. A composition comprising:
about 0.01% w/w tretinoin,
about 1% w/w clindamycin, and
about 4% w/w azelaic acid.

67. A method for treating or preventing acne in a subject in need thereof comprising administering to the skin of the subject any one of the compositions of claims 1-66.

68. A method for treating or preventing photoaging in a subject in need thereof comprising administering to the skin of the subject any one of the compositions of claims 1-66.

69. The method of claim 67 or 68, wherein the composition is administered once per day.

70. A kit comprising:
a) the composition of any one of claims 1-66;
b) a scaled container for housing the composition; and
c) instructions for use.
